(19) United States
(12) Patent Application Publication
(10) Pub. No.: US 2024/0206811 A1
(43) Pub. Date: Jun. 27, 2024

(54) MONITORING SYSTEM, A WEARABLE DEVICE, A NETWORK AND METHODS FOR ACTIVATING A SENSOR IN A WEARABLE DEVICE COMMUNICATIVELY CONNECTED TO THE NETWORK AND CARRIED BY AN INDIVIDUAL

(71) Applicant: Pink Nectarine Health AB, Stockholm (SE)

(72) Inventor: Martin LINREY, Sollentuna (SE)

(73) Assignee: Pink Nectarine Health AB, Stockholm (SE)

(21) Appl. No.: 18/575,446

(22) PCT Filed: Jul. 5, 2022
(86) PCT No.: PCT/SE2022/050681

§ 371 Date: Dec. 29, 2023

(30) Foreign Application Priority Data
Jul. 7, 2021 (SE) 2150907-0

(51) Int. Cl. A61B 5/00; A61B 5/11; H04W 84/18

(52) U.S. Cl. CPC A61B 5/6802; A61B 5/1113; H04W 84/18

(57) ABSTRACT

A monitoring system, a wearable device, a network and methods for activating a sensor in a wearable device carried by an individual and connected to the network are provided. More specifically, a system for monitoring individuals, and a wearable device for activating a sensor of the wearable device in response to input data and/or sensor data obtained by the wearable device is indicating certain events or changes in the behavior or condition of the individual carrying the wearable device.

A monitoring system, a wearable device, a network and methods for activating a sensor in a wearable device communicatively connected to the network and carried by an individual

### TECHNICAL FIELD

The present invention relates to methods, a monitoring system, a node network and energy-efficient wearable devices. Specifically, the present invention relates to a monitoring system, a node network, a wearable device, and methods for providing reliable and fast responses to data obtained by the wearable device is indicating changes in the behavior or condition of an individual/patient carrying a wearable device connected to the network.

### BACKGROUND

Systems for monitoring such individuals, e.g. elderly, sick or injured people, and automatically issuing alarms have been used for some time. Monitoring systems for monitoring such individuals often comprise a personal device (e.g. a wristband) comprising sensors, the signals of which are analysed to determine whether help is needed, e.g. due to a fall.

U.S. Pat. No. 6,433,690 discloses a method and system for recording acceleration and body position data from elderly or disabled persons. The fall monitoring system includes signal feature extraction and interpretive methods for characterizing accelerations and body positions during fall events. The system can detect health and life-threatening fall events in elderly persons and can autonomously notify nursing personnel or family members that the person is in need of immediate assistance. The monitoring of a person's fall is performed by using an accelerometer in a monitoring device carried on the person, which monitoring device samples the person's body angle and body acceleration.

U.S. Pat. No. 9,668,297 describes a BLE scatternet system and method that uses a schedule for when advertisements should be transmitted. The advertiser transmits an initial advertisement, receives a connection request, and then goes to sleep to wake up and transmit subsequent advertisements according to the schedule.

U.S. Ser. No. 10/313,866 describes a method for establishing and monitoring a Bluetooth low energy wireless network by using the broadcast state.

US2019/0215244 describes a Self-Organizing Network (SON) for Internet of Things (IoT), where an IoT device may be selected to be an “IoT coordinator”. A coordinator may e.g. perform the roles of managing a neighboring IoT device and forwarding a packet. This SON method includes receiving neighboring device information from at least one neighboring IoT device of an IoT device, updating device information of the IoT device based on the received neighboring device information, and determining whether the IoT device is a coordinator candidate based on at least one of a number of network interfaces, a number of neighbor links connected to the network interfaces, or a ratio of remaining energy, which is included in the device information.

The managing of the IoT devices in US2019/0215244 is performed by neighboring IoT devices and, thus, relies on the presence of other IoT devices within short-range communication range and is also sharing short-range wireless transmission medium and protocol with other IoT devices thus is dependent on the currently available short-range bandwidth of those neighboring IoT devices, e.g. the current number of other links connected to the network interface of those neighboring IoT devices.

### Problems with the Prior Art

It is difficult to design a system for monitoring wearable devices carried by individuals moving between different locations or rooms of a facility which is highly scalable and provides reliable and fast responses to obtained data which indicates changes in the behavior or condition of an individual/patient, yet provides a battery-efficient solution which is robust in that the wearable devices always have a reliable short-range wireless connection to the node network and compete less for bandwidth.

There is a need for a scalable and monitoring system and energy-efficient wearable devices for monitoring individuals within a facility that also are adapted for providing fast responses to certain events and changes in the behavior or condition of an individual/patient.

More specifically, there is a need for a more scalable and low-power consuming monitoring network and low-power consuming wearable devices enabling for wearable devices connected to the network and worn by monitored individuals, e.g. patients, to provide more detailed sensor data to the network and/or a backend system in response to events and obtained data is indicating certain events and changes in the behavior or condition of an individual/patient, yet provides for the individuals to be connected to the monitoring network while moving between different locations of a facility communicatively covered by different network nodes of the network.

### SUMMARY

The technology disclosed relates to a monitoring system, a node network, a wearable device, and methods for providing reliable and fast responses to data obtained by a wearable device connected to the network is indicating changes in the behavior or condition of an individual/patient carrying the wearable device.

More specifically, the technology disclosed provides a more scalable monitoring and node network, e.g. a self-organizing network of nodes, and energy-efficient wearable devices enabling for wearable devices connected to the network and worn by monitored individuals, e.g. patients, to provide other or more detailed sensor data to the network and/or a backend system in response to events and obtained data is indicating certain events and changes in the behavior or condition of an individual/patient carrying a wearable device, yet provides for the individuals to be connected to the monitoring network while moving between different locations of a facility communicatively covered by different network nodes of the network.

In aspects, the technology disclosed provides a scalable monitoring system and network, and energy-efficient wearable devices for monitoring individuals within a facility that are adapted for providing fast responses to certain events and changes in the behavior or condition of an individual/patient carrying a wearable device by triggering the activation of an inactive energy-consuming sensor or group of sensors of the wearable device to begin obtaining sensor data from the individual and then transmit the obtained sensor data to the network and/or a backend system communicatively coupled to the network.

In aspects, the technology disclosed provides for a system and network adapted for monitoring wearable devices carried by individuals moving between different locations or rooms of a facility which is highly scalable and provides reliable and fast responses to obtained input data, event data and/or sensor data is indicating that an individual carrying a wearable device should activate a sensor or group of sensor that currently is/are inactive, yet provides a battery-efficient solution in that a sensor of the wearable device is activated and consumes power only when obtained input data, event data and/or sensor data is indicating certain events and changes in the behavior or condition of an individual/patient carrying a wearable device, e.g. an alert state, a changed physical condition, a changed health state, a changed physical orientation of the person wearing the wearable device.

The technology disclosed furthermore provides for a system and network which is robust and provides fast responses to events and data obtained by the wearable devices in that the energy-efficient wearable devices always have a reliable short-range wireless connection to the node network and in that the wearable devices compete less for bandwidth with other wearable devices, yet enables the provision of further sensor data and more detailed analyses of the behavior and condition of the individual/patient carrying a wearable device in response to obtained input data and/or sensor data is indicating certain events and changes in the behavior or condition of an individual/patient carrying the wearable device.

In aspects and embodiments, the technology disclosed provides for a more scalable system, a network and methods enabling for wearable devices connected to the network and worn by monitored individuals, e.g. patients, which provides both faster responses to events and obtained input data and/or sensor data indicating to the nodes of the network and/or a backend system is indicating certain events or changes in the behavior or condition of the individual carrying the wearable device, e.g. indicating at least one of an alert state, a changed physical condition, a changed health state, a changed physical orientation of the person wearing the wearable device, yet provides for the individuals to be connected to the monitoring network while moving between different locations of a facility covered by the different network nodes of the network.

In aspects and embodiments, the technology disclosed provides for a system for monitoring individuals within predetermined facilities by providing a backend system which is configured to, in response to input data and/or sensor data obtained by a wearable device is indicating certain events or changes in the behavior or condition of the individual carrying the wearable device, e.g. indicating at least one of an alert state, a changed physical condition, a changed health state, a changed physical orientation of the person wearing the wearable device, initiating the activation of a sensor or group of sensors of the wearable device that currently is/are inactive.

In aspects, the technology disclosed provides for a system for monitoring individuals within predetermined facilities by providing a network node which is configured to, in response to input data and/or sensor data obtained by a wearable device is indicating certain events or changes in the behavior or condition of the individual carrying the wearable device, e.g. indicating at least one of an alert state, a changed physical condition, a changed health state, a changed physical orientation of the person wearing the wearable device, initiating the activation of a sensor or group of sensors of the wearable device that currently is/are inactive.

In aspects and embodiments, the technology disclosed relates to a system for monitoring individuals including a backend system, a self-organizing network (SON) comprising at least three network nodes communicatively connected to each other via established communication links and at least one wearable device comprising at least one sensor, and configured to transmit short-range wireless broadcast signals via its short-range wireless transmitter and temporarily listen for responses to the short-range wireless broadcast signals transmitted by themselves, wherein one of the network nodes is configured to receive and collect input data and/or sensor data for a wearable device which said network node receives from the other network nodes, and
wherein the wearable device is configured to receive control data from said network node and, in response to receiving, from said network node, control data instructing the wearable device to active an inactive sensor or group of sensors of the wearable device:
activate an inactive sensor or group of sensors of the wearable device to start obtaining sensor data from the activated sensor or group of sensors and then transmit the obtained sensor data over an established one short-range wireless communication link with one of the network nodes currently having a bidirectional short-range wireless communication link with the wearable device so that the sensor data from said activated sensor or group of sensors is received by said network node.

In embodiments of the technology disclosed, the wearable device is configured to activate a second sensor or second group of sensors of the wearable device in response to receiving control data from a network node following the uplink transmission, from the SON to the backend system, of at least one of input data and sensor data obtained by a first sensor or first group of sensors of the wearable device different from the second sensor or second group of sensors is indicating to the network node and/or the backend system an alert state, a changed movement pattern or position, a changed physical condition, a changed health state and a changed physical orientation of the person wearing the wearable device. In embodiments, the wearable device may then be configured to activate the second sensor or second group of sensors of the wearable device in response to receiving control data from a network node of the network which has been assigned to be responsible for collecting input data and sensor data from the wearable device.

In embodiments of the technology disclosed, the wearable device is configured to activate a second sensor of the wearable device in response to receiving control data from a network node following the uplink transmission, from the SON to the backend system, of sensor data obtained by at least one first sensor of the wearable device is indicating to the network node and/or the backend system an alert state, a changed movement pattern or position, a changed physical condition, a changed health state and a changed physical orientation of the person wearing the wearable device. In embodiments, the wearable device may then be configured to activate the second sensor of the wearable device in response to receiving control data from a network node of the network which is responsible for collecting sensor data from the wearable device. The network node may then be a network node which has been assigned to be responsible for collecting sensor data from the wearable device, e.g. an assigned controller node for the wearable device.

In embodiments of the technology disclosed, the wearable device is comprising input means and is configured to activate a sensor of the wearable device in response to receiving control data from a network node following the uplink transmission, from the SON to the backend system, of input data obtained by the input means of the wearable device is indicating to the backend system an alert state, a changed movement pattern or position, a changed physical condition, a changed health state and a changed physical orientation of the person wearing the wearable device. In embodiments, the wearable device may then be configured to activate the second sensor of the wearable device in response to receiving control data from a network node of the network which has been assigned to be responsible for collecting the input data from the wearable device, e.g. a controller node for the wearable device. In embodiments, the backend system may be configured to transmit control data or sensor data to the controller node triggering the controller node to, in turn, transmit control data to the wearable device it is responsible for, wherein the control data received from the controller node is triggering the wearable device to activate the second sensor of the wearable device.

In embodiments, the network node is configured to transmit the control data in response to receiving, by the network node, at least one of input data and sensor data obtained by input means and/or at least one sensor of the wearable device which is indicating at least one of an alert state, a changed movement pattern or position, a changed physical condition, a changed health state and a changed physical orientation of the person wearing the wearable device. The network node may then be the network node which is responsible for collecting sensor data from the wearable device, e.g. an assigned controller node for the wearable device. In embodiments, the backend system may be configured to transmit control data or sensor data to the controller node triggering the controller node to, in turn, transmit control data to the wearable device it is responsible for, wherein the control data received by from the controller node is triggering the wearable device to activate at least one sensor of the wearable device.

In embodiments, the network node is configured to transmit the control data in response to receiving, by the network node from the backend system, at least one of control data and sensor data which is at least one of instructing the activation of the at least one sensor and is indicating to the network node at least one of an alert state, a changed movement pattern or position, a changed physical condition, a changed health state and a changed physical orientation of the person wearing the wearable device. The network node may then be the network node which is responsible for collecting sensor data from the wearable device, e.g. an assigned controller node for the wearable device. In embodiments, the backend system may be configured to transmit control data or sensor data to the controller node triggering the controller node to, in turn, transmit control data to the wearable device it is responsible for, wherein the control data received from the controller node is triggering the wearable device to activate at least one sensor of the wearable device.

In embodiments, the network node is configured to receive and collect input data and/or sensor data obtained by the input data and/or sensor data of the wearable device and, in response receiving input data and/or sensor data indicating at least one of an alert state, a changed movement pattern or position, a changed physical condition, a changed health state and a changed physical orientation of the person wearing the wearable device:
transmit control data to the wearable device instructing the wearable device to activate a sensor of the wearable device.

In embodiments, the network node is configured to configured to receive and collect at least one of input data and sensor data obtained by an input means and/or sensor of the wearable device and, in response to the receiving input data and/or sensor data indicating at least one of an alert state, a changed movement pattern or position, a changed physical condition, a changed health state and a changed physical orientation of the person wearing the wearable device:
transmit control data to the wearable device instructing the wearable device to at least one of: start discarding sensor data obtained by a sensor of the wearable device, change the amount of discarded sensor data obtained by a sensor of the wearable device, and increase or decrease the sampling rate or interval for at least one sensor of the wearable device.

In aspects and embodiments, the technology disclosed relates to a system for monitoring individuals including a backend system, a self-organizing network (SON) comprising at least three network nodes communicatively connected to each other via established communication links and at least one wearable device comprising at least one sensor, and configured to transmit short-range wireless broadcast signals via its short-range wireless transmitter and temporarily listen for responses to the short-range wireless broadcast signals transmitted by themselves,
wherein a wearable device is assigned one network node to have the controller node role for the wearable device,
wherein the network node having the controller node role for a wearable device is configured to receive and collect input data and/or sensor data for the wearable device received from the other network nodes, and
wherein the wearable device is configured to receive control data from the network node having the controller node role for the wearable device and,
in response to receiving, from the network node having the controller node role for the wearable device, control data instructing the wearable device to active an inactive sensor or group of sensors of the wearable device:
   activate an inactive sensor or group of sensors of the wearable device to start obtaining sensor data from the activated sensor or group of sensors and then transmit the obtained sensor data over an established one short-range wireless communication link with one of the network nodes currently having a bidirectional short-range wireless communication link with the wearable device so that the sensor data from said activated sensor or group of sensors is received by the network node having the controller node role for the respective wearable device.

In aspects and embodiments, the technology disclosed relates to a system for monitoring individuals including a backend system, a self-organizing network (SON) comprising at least three network nodes communicatively connected to each other via established communication links and at least one wearable device comprising at least one sensor, and configured to transmit short-range wireless broadcast signals via its short-range wireless transmitter and temporarily listen for responses to the short-range wireless broadcast signals transmitted by themselves,
wherein each wearable device is assigned one network node to have the controller node role for the wearable device in that the controller node for a wearable device,
wherein the network node having the controller node role for a wearable device is further configured to receive and collect input data and/or sensor data for the wearable device received from the other network nodes, and
wherein each wearable device is further configured to receive control data from the network node having the controller node role for the respective wearable device and,
in response to receiving, from the network node having the controller node role for the wearable device, control data instructing the wearable device to active an inactive sensor or group of sensors of the wearable device:
   activate an inactive sensor or group of sensors of the wearable device to start obtaining sensor data from the activated sensor or group of sensors and then transmit the obtained sensor data over the established one short-range wireless communication link with the only one network node currently having a bidirectional short-range wireless communication link with the wearable device so that the sensor data from said activated sensor or group of sensors is received by the network node having the controller node role for the respective wearable device.

In aspects and embodiments, the technology disclosed relates to a system for monitoring individuals including a backend system, a self-organizing network (SON) comprising at least three network nodes communicatively connected to each other via established communication links and at least one wearable device comprising at least one sensor, and configured to transmit short-range wireless broadcast signals via its short-range wireless transmitter and temporarily listen for responses to the short-range wireless broadcast signals transmitted by themselves,
wherein each wearable device is assigned one network node to have the controller node role for the wearable device in that the controller node for a wearable device is configured to control and dynamically change which only one of the network nodes that currently has an established bidirectional short-range wireless communication link with the wearable device,
wherein the network node having the controller node role for a wearable device is further configured to receive and collect input data and/or sensor data for the wearable device received from the other network nodes, and
wherein each wearable device is further configured to receive control data from the network node having the controller node role for the respective wearable device and, in response to receiving, from the network node having the controller node role for the wearable device, control data instructing the wearable device to active an inactive sensor or group of sensors of the wearable device:
   activate an inactive sensor or group of sensors of the wearable device to start obtaining sensor data from the activated sensor or group of sensors and then transmit the obtained sensor data over the established one short-range wireless communication link with the only one network node currently having a bidirectional short-range wireless communication link with the wearable device so that the sensor data from said activated sensor or group of sensors is received by the network node having the controller node role for the respective wearable device.

In aspects and embodiments, the technology disclosed provides for a system for monitoring individuals within predetermined facilities by providing a self-organizing network (SON) of network nodes, e.g. hub nodes, including hub nodes configured for having the controller node role for a particular wearable device in being responsible for collecting input data and/or sensor data obtained by the wearable device and thereby being configured to, in response to the obtained input data and/or sensor data is indicating certain events or changed behavior or condition of the individual carrying the wearable device, e.g. indicating at least one of an alert state, a changed physical condition, a changed health state, a changed physical orientation of the person wearing the wearable device, initiating the activation of a sensor or group of sensors of the wearable device the respective network node is controller node for.

In aspects, the technology disclosed relate to a system for monitoring individuals including a backend system, a self-organizing network (SON) comprising at least three network nodes including at least one hub node configured to receive and collect data from other network nodes, at least two satellite nodes configured to both transmit short-range wireless broadcast signals and listen for short-range wireless broadcast signals transmitted by other devices and nodes, and at least one wearable device comprising at least one of input means and at least one sensor, and configured to transmit short-range wireless broadcast signals via its short-range wireless transmitter and temporarily listen for responses to the short-range wireless broadcast signals transmitted by themselves, wherein each wearable device is assigned one hub node to have the controller node role for the wearable device in that the controller node for a wearable device is configured to control and dynamically change which only one of the network nodes that currently have a short-range wireless connection with the wearable device, wherein the network node having the controller node role for a wristband is further configured to receive and collect input data and/or sensor data obtained by the wearable device and which may be received from other network nodes of the SON, and wherein each wearable device is configured to receive or obtain at least one of control data, input data and sensor data and, in response to receiving or obtaining at least one of control data, input data and sensor data indicating to the wearable device at least one of an alert state, a changed movement pattern or position, a changed physical condition, a changed health state and a changed physical orientation of the individual wearing the wearable device, activate an inactive sensor or group of sensors of the wearable device to start obtaining sensor data from the activated sensor or group of sensors and then transmit the obtained sensor data over the established one short-range wireless connection and via the SON so that the sensor data is received by the network node having the controller node role for the wearable device.

In embodiments, the wearable devices of the technology disclosed are configured to activate a second sensor or group of sensors of the wearable device in response to at least one of sensor data obtained by a first sensor or group of sensors of the wearable device indicating at least one of an alert state, a changed movement pattern or position, a changed physical condition, a changed health state and a changed physical orientation of the person wearing the wearable device and control data received from the controller node for the wearable device and which transmission was triggered by sensor data obtained by the first sensor or group of sensors of the wearable device indicating at least one of an alert state, a changed movement pattern or position, a changed physical condition, a changed health state and a changed physical orientation of the person wearing the wearable device.

In embodiments, a hub node having the controller node role for a wearable device and being responsible for the wearable device is further configured to receive and collect input data and/or sensor data obtained by the input data and/or sensor data of the wearable device and, in response receiving input data and/or sensor data indicating at least one of an alert state, a changed movement pattern or position, a changed physical condition, a changed health state and a changed physical orientation of the person wearing the wearable device:
transmit control data to the wearable device instructing the wearable device to activate the sensor of the wearable device.

In embodiments, a hub node having the controller node role for the wearable device configured to receive and collect at least one of input data and sensor data obtained by an input means and/or sensor of the wearable device and, in response to the receiving input data and/or sensor data indicating at least one of an alert state, a changed movement pattern or position, a changed physical condition, a changed health state and a changed physical orientation of the person wearing the wearable device:
transmit control data to the wearable device instructing the wearable device to at least one of: start discarding sensor data obtained by a sensor of the wearable device, change the amount of discarded sensor data obtained by a sensor of the wearable device, and increase or decrease the sampling rate or interval for at least one sensor of the wearable device.

In embodiments, the activated sensor or group of sensors is in sleep mode operation before being activated by the wearable device.

In embodiments, the downlink data received from the backend system is included in at least one of a configuration file which is continuously updated, by the backend system based on sensor data and/or sensor data received by the backend system from the SON/the controller nodes, and then transmitted from the backend system to the SON. The configuration file may then be transmitted as control data transmitted downlink from the backend system to the SON. In various embodiments of the technology disclosed, the control data received from the backend system may in itself be adapted for indicating to a controller node for a wearable device that a currently inactive sensor of the wearable device should be activated the activation or the configuration file may only provide the controller nodes with continuously updated sensor data and/or input data and the network nodes having the controller node role for the respective wearable device may be configured to determine, based on the updated sensor data and/or input data in the configuration file, that a sensor should be activated. After receiving the instructions from the backend system or determining that a sensor should be activated, the network node having the controller node role for a wearable device may then be further configured to transmit control data to the wearable device instructing the wearable device to activate a currently inactive sensor to start obtaining sensor data which is transmitted at least one of uplink to the controller node via the established short-range wireless communication link with the connection node and in short-range wireless broadcast signals that may be received by any network node within short-range wireless communication range.

In embodiments, a hub node having the controller node role for a wearable device is configured to, in response to receiving downlink data from the backend system which indicates at least one of an alert state, a changed physical condition, a changed health state, a changed physical orientation of the person wearing the wearable device and that a call should be established with the wearable device: transmit control data to the wearable device instructing the wearable device to activate a currently inactive sensor or group of sensors of the wearable device. The downlink data may then be based on at least one of input data and sensor data obtained by the wearable device which is transmitted to the backend system by the one hub node assigned by the SON to have the controller node role for the wearable device. The downlink data received from the backend system may then be comprised in at least one of a configuration file which is continuously updated by the backend system and which is based on input data and/or sensor data received from the SON and then transmitted from the backend system to the SON, and control data transmitted downlink from the backend system to the SON which is based on input data and/or sensor data received from the SON and which is adapted for instructing the hub node having the controller node role for the wearable device to initiate the transmission of the control data to the wearable device instructing the wearable device to activate at least one currently inactive sensor of the wearable device.

In embodiments, a hub node having the controller node role for a wearable device is further configured to, in response to receiving downlink data originating from the backend system and which indicates at least one of an alert state, a changed physical condition, a changed health state, a changed physical orientation of the person wearing the wearable device and that a call should be established with the wearable device, transmit control data instructing the wearable device to activate the currently inactive sensor or group of sensors of the wearable device. The backend system may then be configured to transmit downlink data to the SON at least partly based on at least one of input data obtained by the input device of the wearable device and sensor data obtained by at least one sensor of the wearable device and which is received by the backend system from the SON. The input data and/or sensor data may then be received by the backend system from the hub node assigned to have the controller node role for the wearable device.

In example embodiments, the system comprises at least two hub nodes and each of the at least one wearable device is assigned, by the SON, one of the hub nodes to have the controller node role for the respective wearable device and thereby configured for dynamically determining and changing which node is currently having the only one short-range wireless communication link established between the SON and the wearable device it is responsible for.

In embodiments, the SON comprises at least two hub nodes each communicatively coupled to the other hub nodes through established connections and configured to receive at least one of input data and sensor data from the at least one wearable device and then share the received data with other hub nodes by distributing the data over the established connections links.

In example embodiments, the SON comprises at least three satellite nodes each configured to establish a short-range wireless connection with a wearable device in response to instructions received from the node having the controller node role for the respective wearable device, and to forward input data and/or sensor data received from the wearable device in short-range wireless broadcast transmissions received so that the input data and/or sensor data can be received and stored by the node having the controller node role for the wearable device.

In aspects and embodiments, the technology disclosed relates to methods, a system comprising a self-organizing network (SON) of nodes and at least one wearable devices configured to transmit input data and/or sensor data obtained by the respective wearable device both via an established short-range wireless connection links with one of the network nodes of the node network and via short-range wireless broadcasts which may be received by any of the network nodes that are within short-range wireless communication range with the wearable device.

The technology disclosed also relates to a system and methods for transmitting input data and/or sensor data obtained by a personal device worn by an individual both via an established short-range wireless connection link between the personal device and a network node and via short-range wireless broadcasts transmitted by the personal device.

In aspects, the personal device is adapted to be worn by an individual and is configured to transmit short-range wireless broadcast signals while a short-range wireless connection between the wearable device and a node of the network of nodes is active, thereby being configured for providing its obtained input data and/or sensor data to other nodes of the network that are within short-range wireless communication range with the personal device.

In aspects and embodiments, the technology disclosed relates to a system including a backend system, a self-organizing network (SON) comprising at least three network nodes and at least one wearable device comprising at least one sensor and/or user input means and configured to transmit short-range wireless broadcast signals and temporarily listen for responses to the short-range wireless broadcast signals transmitted by themselves. In this embodiment, each of the at least one wearable device is assigned one hub node to have the controller node role for the wearable device in that the node is configured to obtain and receive sensor data and/or user input data obtained by the wearable device it is the controller node for and, in response to the sensor data and/or user input data obtained by the wearable device is indicating changes in the behavior or the medical condition of the individual carrying the wearable device, transmit control data to the wearable device instructing the wearable device to active an inactive sensor of the wearable device. In this embodiment, the wearable device thus activates the inactive sensor based on the control data received from the network node having the controller node role for the wearable device and then begins transmitting sensor data obtained by the activated sensor at least over an established short-range wireless communication link, e.g. Bluetooth link, with one of the network nodes. The controller node may then use input data and/or sensor obtained or received by the controller node to determine that a sensor is to be activated, e.g. sensor data from a body temperature sensor and/or movement sensor data detected by the wearable device may be used in combination by the backend system to determine changes in the behaviour and medical condition of the individual carrying the wearable device and that a currently inactive power-consuming blood pressure sensor of the wearable device should be activated. In response to determining certain changes in the behaviour and the medical condition of the individual carrying the wearable device that trigger the transmission of control data, the controller node for the wearable device then transmits the control data to the wearable device instructing the wearable device to active the inactive sensor of the wearable device.

In certain embodiments, processing means of the controller node uses a trained machine learning algorithm to analyse the data associated with the wearable device and which is collected or received by the controller node, since machine learning is an efficient way of evaluating obtained sensor data and event data, e.g. in situations where large amounts of data is collected. In embodiments, the trained machine learning algorithm of the controller node may then use obtained input data and/or sensor to determine that a sensor is to be activated, e.g. sensor data from a body temperature sensor and/or movement sensor data detected by the wearable device may be used by the trained machine learning algorithm to determine changes in the behaviour and condition of the individual carrying the wearable device and that a power-consuming blood pressure sensor of the wearable device should be activated.

In aspects and embodiments, the technology disclosed relates to a system including a backend system, a self-organizing network (SON) comprising at least three network nodes and at least one wearable device comprising at least one sensor and/or user input means and configured to transmit short-range wireless broadcast signals and temporarily listen for responses to the short-range wireless broadcast signals transmitted by themselves. Each of the at least one wearable device is assigned one hub node to have the controller node role for the wearable device in that the node is configured to obtain and receive sensor data and/or user input data obtained by the wearable device it is the controller node for and then forward at least some of the data obtained by the wearable device to the backend system. In this embodiment, the hub node having the controller node role for a wearable device receives data (at least partly based on the data previously forwarded to the backend system) associated with the wearable device from the backend system and, in response to the data received from the backend system is at least one of instructing the controller node to transmit or forward control data and indicating changes in the behavior or medical condition of the individual carrying the wearable device, transmit or forward control data to the wearable device instructing the wearable device to active an inactive sensor of the wearable device. In this embodiment, the wearable device thus activates the inactive sensor based on the control data received from the network node having the controller node role for the wearable device and then begins transmitting sensor data obtained by the activated sensor at least over an established short-range wireless communication link, e.g. Bluetooth link, with one of the network nodes. The backend system may then use input data and/or sensor received from the controller node to determine that a sensor is to be activated, e.g. sensor data from a body temperature sensor and/or movement sensor data detected by the wearable device may be used in combination by the backend system to determine changes in the behaviour and medical condition of the individual carrying the wearable device and that a currently inactive power-consuming blood pressure sensor of the wearable device should be activated.

In certain embodiments, processing means of the backend system uses a trained machine learning algorithm to analyse the data associated with the wearable device and which is received from the controller node, since machine learning is an efficient way of evaluating obtained sensor data and event data, e.g. in situations where large amounts of data is collected. In embodiments, the trained machine learning algorithm of the backend system may then use input data and/or sensor received from the controller node to determine that a sensor is to be activated, e.g. sensor data from a body temperature sensor and/or movement sensor data detected by the wearable device may be used by the trained machine learning algorithm to determine changes in the behaviour and condition of the individual carrying the wearable device and that a power-consuming blood pressure sensor of the wearable device should be activated.

In certain embodiments, processing means of the wearable device may use a trained machine learning algorithm to analyse the sensor data obtained by at least one sensor of the wearable device, since machine learning is an efficient way of evaluating obtained sensor data and event data, e.g. in situations where large amounts of data is collected. In embodiments, the trained machine learning algorithm of the wearable device may then use obtained input data and/or sensor to determine that a sensor is to be activated, e.g. sensor data from a pressure sensor and/or movement sensor data of the wearable device may be used by the trained machine learning algorithm to determine changes in the behaviour and condition of the individual carrying the wearable device and that a power-consuming body temperature sensor of the wearable device should be activated.

In aspects, one of the hub nodes is assigned to have the controller node role for a wearable device and be responsible for collecting input data and/or sensor data obtained by the wearable device. The input data and/or sensor data obtained by the wearable device may be received in broadcast messages transmitted by the wearable device if the controller node is within short-range wireless communication range with the wearable device or may be received from other network nodes within the SON, including satellite nodes which are configured to both transmit short-range wireless broadcast signals and listen for short-range wireless broadcast signals transmitted by other devices and nodes.

In embodiments, the hub nodes of the SON are communicatively connected to each other via a IP-based network such as a wifi and each of the hub nodes are configured to share data associated with any of the wearable devices via continuous distribution of the data within the IP-based network.

In embodiments, the hub nodes of the SON are communicatively connected to each other via a network of established short-range wireless communication links established between the hub nodes, e.g. established Bluetooth or BLE connections, and each of the hub nodes are configured to share data associated with any of the wearable devices via continuous distribution of the data within the network of established short-range wireless communication links, e.g. BLE links.

In aspects, each of the satellite nodes may have an established short-range wireless communication link, e.g. BLE link, with one of the wearable devices, where the establishment of the short-range wireless communication link is initiated by the hub node of the SON which is the controller node for the wearable device.

The technology disclosed relates to a system including a backend system, a self-organizing network (SON) comprising:
at least three network nodes including at least one hub node and at least two satellite nodes each comprising a speaker and configured to transmit short-range wireless broadcasts and to establish a short-range wireless communication link with a wearable device, and at least one wearable device having short-range wireless communication capabilities and comprising a microphone and at least one of at least one sensor and user input means,
   wherein each of the at least one wearable device is having an established short-range wireless communication link with one of the network nodes and is assigned a hub node to have the role of a controller node for the wearable device, and wherein the hub node having the controller node role for a wearable device is configured to, in response to receiving downlink data from the backend system indicating certain changes in the behavior, movement pattern or medical conditions of the individual carrying the wearable device, initiate the transmission of control data to the wearable device instructing the wearable device to active a sensor that currently is inactive and begin transmitting sensor data obtained by the activated sensor.

The technology disclosed relates to a system including a backend system, a self-organizing network (SON) comprising:
at least three network nodes including at least one hub node and at least two satellite nodes each comprising a speaker and configured to transmit short-range wireless broadcasts and to establish a short-range wireless communication link with a wearable device, and
at least one wearable device having short-range wireless communication capabilities and comprising a microphone and at least one of at least one sensor and user input means,
   wherein each of the at least one wearable device is having an established short-range wireless communication link with one of the network nodes and is assigned a hub node to have the role of a controller node for the wearable device, and wherein the hub node having the controller node role for a wearable device is configured to, in response to obtaining or receiving input data and/or sensor data obtained by a user input means or sensor of the wearable device indicating certain changes in the behavior, movement pattern or medical conditions of the individual carrying the wearable device, initiate the transmission of control data to the wearable device instructing the wearable device to active a sensor that currently is inactive and begin transmitting sensor data obtained by the activated sensor.

In embodiments, the backend system is configured to transmit the downlink data to the network/controller node in response to receiving uplink data from the SON indicating to the backend system at least one of an alert state, a changed physical condition, a changed health state, a changed physical orientation of the person wearing the wearable device and that a call should be established with the wearable device.

In embodiments, the downlink data received by the SON and the controller node is included in at least one of a configuration file which is continuously updated by the backend system and transmitted to the SON, where the data in the configuration file is based on sensor data obtained by the wearable device and transmitted to the backend system by the network node having the controller node role for the wearable device.

In embodiments, the backend system is configured to continuously update data associated with the at least one wearable device based on input data and/or sensor data received by the backend system from the SON, and wherein the downlink data which may trigger the activation of a sensor of a wearable device is included in a configuration file continuously updated and transmitted by the backend system to the SON.

In embodiments, the network node currently having an established wireless short-range communication link with the wearable device may be configured to at least one of transmit and forward a request to the wearable device requesting the wearable device to activate its sensor and then transmit sensor data obtained by the activated sensor over the already established wireless short-range communication link with the wearable device.

In embodiments, the technology disclosed relates to a method in a system including a network comprising at least three network nodes and at least one wearable device having short-range wireless communication capabilities and comprising at least one sensor and a user input means, the method comprising:
a. assigning one of the network nodes to be responsible for a particular wearable device in collecting sensor data and/or input data obtained and transmitted by the wearable device to the network nodes of the network;
b. continuously receiving, by the network node responsible for a wearable device, sensor data and/or user input data obtained by the at least one sensor and a user input means of the wearable device;
c. initiating, by the network node responsible for the wearable device and in response to certain received sensor data and/or user input data is indicating to the network node certain changes in the behavior or medical condition of the individual carrying the wearable device and instructions that a sensor of the wearable device is to be activated, the activation of a sensor of the wearable device.

In embodiments, the technology disclosed relates to a method in a system including a backend system, a network comprising at least three network nodes and at least one wearable device having short-range wireless communication capabilities and comprising at least one sensor and a user input means, the method comprising:
a. assigning one of the network nodes to be responsible for a wearable device in collecting sensor data and/or input data obtained and transmitted by the wearable device to the network nodes of the network;
b. continuously receiving, by the network node responsible for a wearable device, sensor data and/or user input data obtained by the at least one sensor and a user input means of the wearable device;
c. transmitting, by the node responsible for the wearable device to the backend system, data based on the received sensor data and/or user input data;
d. transmitting, by the backend system, downlink data to the SON, wherein the downlink data is based on sensor data and/or user input data received from the node responsible for the wearable device;
e. receiving, by the node responsible for the wearable device, the downlink data from the backend system;
f. initiating, by the network node responsible for the wearable device and in response to the downlink data from the backend system is containing at least one of data indicating to the responsible network node certain changes in the behavior or medical condition of the individual carrying the wearable device and instructions that a sensor of the wearable device is to be activated, the activation of a sensor of the wearable device by:
   g. transmitting, by the node responsible for the wearable device to the wearable device, control data instructing the wearable device to active a sensor.

In embodiments, each of the plurality of personal devices is configured to transmit data indicating the same obtained sensor data and/or event data both via their respective established short-range wireless connection with their respectively assigned connection node and via short-range wireless broadcast signals. The same sensor data and/or event data may then be data indicating at least one of the activation of an input means of the personal device, data indicating a trigger event and sensor data indicating a health state and/or alert state for the personal device and/or the individual wearing the personal device.

In embodiments, the controller node for the respective personal device is configured to determine a specific alert state and/or a health state for the individual wearing the personal device at least partly based on received sensor data and/or event data associated with the personal device.

In certain embodiments, the controller node for the respective personal device is configured to set or change a health state for the individual wearing a personal device at least partly based on received sensor data obtained by and/or originating from at least one medical sensor of the personal device.

In embodiments, the controller node currently assigned to a personal device is configured to transmit alert state data and/or alarm data to a local or remote processing arrangement, e.g. backend system, based on received sensor data and/or event data. The local or remote processing arrangement may then be configured to set an alarm or determine whether to set an alarm based on the received alert state data and/or alarm data by informing and/or transmitting an alarm to the staff at the facility, e.g. send an alarm message to the mobile communication devices belonging to the staff members at the facility.

In embodiments, the controller node currently assigned to a personal device is configured to transmit alert state data and/or alarm data to a local or remote processing arrangement, e.g. backend system, based on received sensor data and/or event data. The local or remote processing arrangement may then be configured to set an alarm or determine whether to set an alarm based on the received alert state data and/or alarm data by informing and/or transmitting an alarm to the staff at the facility, e.g. send an alarm message to the mobile communication devices belonging to the staff members at the facility.

In embodiments, each of the network nodes of the node network is configured to first measure signal strengths of short-range wireless broadcast signals received from a personal device and then automatically transmit or forward data indicating the measured signal strengths to the identified controller node responsible for the personal device transmitting the short-range wireless broadcast signals.

In embodiments, the controller node currently assigned to a personal device is configured to determine a position and/or a location for their respective personal device at least partly based on the received data indicating the measured signal strengths.

In embodiments, the controller node for the respective personal device is configured to set an alert state or alarm data based on received sensor data and/or event data and also determine a position and/or a location for their respective personal device at least partly based on the received data indicating the measured signal strengths.

In embodiments, the controller node for the respective personal device is configured to transmit alert state data and/or alarm data to a local or remote processing arrangement, e.g. backend system, based on received sensor data and/or event data and also determine a position and/or a location for their respective personal device at least partly based on the received data indicating the signal strengths of short-range wireless broadcast signals measured by the network nodes within short-range wireless communication range with the personal device transmitting the broadcast signals. The local or remote processing arrangement may then set an alarm or determine whether to set an alarm based on the received alert state data and/or alarm data by informing and/or transmitting an alarm to the staff at the facility where the alarm includes the determined position and/or location, e.g. send an alarm message to the mobile communication devices belonging to the staff members at the facility.

In embodiments, the controller node for the respective personal device is configured to set or change a health state for the individual wearing a personal device at least partly based on received sensor data obtained by and/or originating from at least one medical sensor of the personal device and also determine a position and/or a location for their respective personal device at least partly based on the received data indicating the measured signal strengths.

In embodiments, the determining of a position and/or location for the individual wearing the personal device by the controller node may be based on the received signal strength indication (RSSI) of short-range wireless broadcast signals transmitted by the personal device and which are received by the network nodes within short-range communication range with the personal device. The network nodes within short-range communication range then measure the RSSI of short-range wireless broadcast signals and forwards the RSSI to the controller node for the personal device. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by a personal device may be used by the controller node assigned to a personal device for determining the current position and/or location for the personal device in connection with transmitting position or location data together with alert state data or when setting an alarm for the individual wearing the personal device. Thus, the decision by the controller node for a particular personal device to transmit alert data or set an alarm for the individual may be based on broadcast signal strength values measured and received from other nodes within short-range communication range with the personal device (including the current controller node and the current connection node for the personal device if any of these two nodes are within short-range wireless communication range with the personal device to receive the broadcast signals).

In aspects and embodiments, the technology disclosed relates to a method in a system including a network comprising network nodes and a personal device with short-range wireless communication capabilities, said method comprising:
a. assigning a network node to be controller node for the personal device;
b. assigning, by said controller node, a connection node for establishing a short-range wireless communication link between said connection node and said personal device;
c. establishing, by said assigned connection node, a short-range wireless communication link with said personal device;
d. transmitting, from said personal device and via said established short-range wireless communication link, a message to said connection node;
e. receiving, by said connection node, said message, wherein said message contains data associated with said personal device;
f. forwarding, from the assigned connection node to the controller node for said personal device, said message or data to said assigned controller node for said personal device; and
g. transmitting, from said personal device, a short-range wireless broadcast signal, wherein said short-range wireless broadcast signal is transmitted while said short-range wireless communication link between said connection node and said personal device is active, and wherein said short-range wireless broadcast signal is received or detected by any network node currently within short-range communication range with said personal device.

In aspects and embodiments, the technology disclosed relates to a method in a system including a network comprising at least two network nodes and a plurality of personal devices with short-range wireless communication capabilities, the method comprising:
a. assigning, e.g. automatically by the at least two network nodes and based on a common decision model which is known to the at least two network nodes, one node among the at least two network nodes to be the controller node for a personal device;
b. assigning a connection node for establishing a short-range wireless communication link between said connection node and said personal device;
c. establishing, by the assigned connection node, a short-range wireless communication link with said personal device;
d. transmitting, from the personal device to the connection node via the established short-range wireless communication link, a message containing at least one of status data, sensor data and event data associated with the personal device;
e. receiving, by the connection node assigned to the personal device, said message;
f. transmitting, from the assigned connection node to the controller node for the personal device, data including said at least one of status data, sensor data and event data associated with the personal device; and
g. transmitting, from said personal device, at least one short-range wireless broadcast signal, wherein said at least one short-range wireless broadcast signal is transmitted while said short-range wireless communication link between said connection node and said personal device is active.

In aspects and embodiments, the technology disclosed relates to a method in a system including a network comprising network nodes and a personal device with short-range wireless communication capabilities, said method comprising:
a. assigning a network node to be controller node for the personal device;
b. assigning, by said controller node, a connection node for establishing a short-range wireless communication link between said connection node and said personal device;
c. establishing, by said assigned connection node, a short-range wireless communication link with said personal device;
d. transmitting, from said personal device and via said established short-range wireless communication link, a message to said connection node;
e. receiving, by said connection node, said message, wherein said message contains data associated with said personal device;
f. forwarding, from the assigned connection node to the controller node for said personal device, said message or data to said assigned controller node for said personal device; and
g. transmitting, from said personal device, a short-range wireless broadcast signal, wherein said short-range wireless broadcast signal contains data associated with said personal device and is transmitted while said short-range wireless communication link between said connection node and said personal device is active, and wherein said short-range wireless broadcast signal is received or detected by any network node currently within short-range communication range with said personal device; and
h. forwarding, by any and all network nodes currently within short-range communication range with said personal device and to said controller node for said personal device, at least one of data extracted from said short-range wireless broadcast signal and measured, by each of the network nodes received the broadcast signal, respective signal strengths of the received short-range wireless broadcast signal.

In embodiments and embodiments, said at least one of status data, sensor data and event data associated with said personal device is provided, e.g. automatically provided, to said controller node by forwarding said received message from said connection node or by transmitting, from said connection node, a message including data corresponding to, or at least partly based on, said received at least one of status data, sensor data and event data associated with said personal device.

In embodiments, said message is forwarded or transmitted via an IP-based network connecting the network nodes.

In embodiments, said IP-based network is a separate network and uses a different communication protocol from the communication protocol used for said established short-range wireless communication link between said connection node and said personal device.

In embodiments, said established short-range wireless communication link between said connection node and said personal device is a bidirectional short-range wireless communication link.

In embodiments, said message from said connection node to said controller node and said at least one short-range wireless broadcast signal transmitted from said personal device are containing data indicating the same sensor data and/or event data.

In embodiments, the message from the connection node to the controller node is containing data indicating at least one of the activation of an input means of the personal device, data indicating a trigger event and sensor data indicating a health state and/or alert state for the personal device and/or the individual wearing the personal device.

In embodiments, the at least one short-range wireless broadcast signal transmitted from the personal device is containing data indicating at least one of the activation of an input means of the personal device, data indicating a trigger event and sensor data indicating a health state and/or alert state for the personal device and/or the individual wearing the personal device.

In embodiments, the message transmitted from the connection node is containing sensor data and/or event data, and the method is further comprising:
a. determining, by the controller node and at least partly based on received sensor data and event data associated with the personal device, a specific alert state and/or a health state for the individual wearing the personal device.

In embodiments, the message transmitted from the connection node is containing sensor data and/or event data, and the method is further comprising:
b. transmitting, from the controller node to a local or remote processing arrangement, at least one of said sensor data and/or event data and data indicating a changed health state and/or alert state for the individual wearing the personal device.

In embodiments, the message transmitted from the connection node is containing sensor data and/or event data, and the method is further comprising:
c. transmitting, from the controller node, the sensor data and/or event data associated with the personal device to a local or remote processing arrangement; and
d. determining, by the local or remote processing arrangement, the current health state and/or alert state for the individual wearing the personal device based on said sensor data and/or event data, wherein processing of the sensor data for determining the current health state and/or alert state is performed by the local or remote processing arrangement using a trained machine learning algorithm.

In embodiments, the method is further comprising:
e. determining, by the local or remote processing arrangement, a changed health state and/or alert state for the individual wearing the personal device controller node, wherein the determining is at least partly based on said received sensor data and event data associated with the personal device is indicating a specific alert state and/or a health state for the individual wearing the personal device; and
f. transmitting, from the controller node to a local or remote processing arrangement, data indicating the determined changed health state and/or alert state for the individual wearing the personal device.

In embodiments, the method is further comprising:
g. setting, by the local or remote processing arrangement, an alarm for the personal device and/or individual wearing the personal device, wherein said setting of an alarm is at least partly based on the determining of a changed health state and/or alert state for the individual wearing the personal device.

In embodiments, the method according to the technology disclosed is comprising:
a. receiving, by at least one network node within short-range wireless communication range with the personal device, at least one short-range wireless broadcast signal;
b. measuring, by each of the at least one network node within short-range wireless communication range with the personal device, the signal strengths of the at least one short-range wireless broadcast signal;
c. transmitting, from each of the at least one network node within short-range wireless communication range with the personal device and to the controller node, at least one message containing data indicating the respective signal strengths of the at least one short-range wireless broadcast signal measured by the respective at least one network node within short-range wireless communication range with the personal device; and
d. receiving, by the controller node for the personal device, at least one message containing data indicating the respective signal strengths, wherein a message is received from the respective of the at least one network node and is indicating the respective signal strengths measured by the respective at least one node within short-range wireless communication range with the personal device.

In embodiments, the method is further comprising:
h. determining, by the controller node and at least partly based on the data indicating measured signal strengths, the current position and/or location for the personal device and/or the individual wearing the personal device.

In embodiments, the determining of the current position and/or location for the personal device and/or the individual wearing the personal device is in addition based on sensor data obtained by a motion sensor and/or pressure sensor of the personal device.

In embodiments, the method is further comprising:
i. transmitting, from the controller node to a local or remote processing arrangement, data indicating the measured signal strengths for the respective at least one node; and
j. determining, by the local or remote processing arrangement, the current position and/or location for the personal device and/or the individual wearing the personal device, wherein said determining is at least partly based on received data indicating measured signal strengths for the respective at least one node.

In embodiments, the method is further comprising:
k. obtaining, by at least one of a motion sensor and a pressure sensor of the personal device, sensor data indicating a pressure and/or a movement associated with the personal device and/or the individual wearing the personal device; and
l. transmitting, from the personal device to the connection node via the established short-range wireless communication link, data indicating said sensor data obtained by the personal device;
m. receiving, by the connection node, data indicating said sensor data obtained by the personal device;
n. forwarding, by the connection node to the controller node, data indicating said sensor data obtained by the personal device; and
o. determining, by the controller node, the current position and/or location for the individual earing the personal device, wherein the determining of the current position and/or location is at least partly based on said sensor data obtained by a motion sensor and/or a pressure sensor of the personal device.

In embodiments, the message from the connection node is containing sensor data and/or event data, and the method is further comprising:
p. transmitting, from the controller node, said sensor data and/or event data associated with the personal device to a local or remote processing arrangement; and
q. determining, by the local or remote processing arrangement, the current health state and/or alert state for the individual wearing the personal device based on said sensor data and/or event data, wherein the processing of the sensor data for determining the current health state and/or alert state is performed by the local or remote processing arrangement using a trained machine learning algorithm.

In embodiments, the method is further comprising:
r. determining, by the controller node, a changed health state and/or alert state for the individual wearing the personal device controller node at least partly based on received sensor data and event data associated with the personal device is indicating a specific alert state and/or a health state for the individual wearing the personal device; and
s. transmitting, from the controller node to a local or remote processing arrangement, data indicating a changed health state and/or alert state for the individual wearing the personal device.

The technology disclosed provides a solution for acting on an identified event by transmitting (redundant) event data, e.g. event/alarm data which is triggered by pressing a button on a wristband or triggered by voice activation of a voice activation means of the personal device, both via an established short-range wireless connection with one of the nodes and via at least one short-range wireless broadcast signal or message, e.g. Bluetooth broadcast signals or messages, from the personal device and where the short-range wireless broadcast signal are received by the network nodes within short-range wireless communication range with the personal device.

In embodiments, the technology disclosed provides a solution for fast response to obtained sensor data or an identified event, e.g. health sensor data indicating a health state, e.g. a deteriorated health condition, of the individual wearing the personal device or the event of pressing an alarm button on a personal device such as a wristband. The fast response solution according to the technology disclosed is achieved by the transmission of sensor data and/or event data indicating health sensor data, e.g. obtained by a medical sensor, or the occurrence of an event such as the pressing an alarm button on the personal device or activating an voice activation means, both via an established short-range wireless connection with one of the network nodes and via short-range wireless broadcast transmissions, e.g. via Bluetooth, from the personal device and which are received by those network nodes which are within short-range wireless communication range, e.g. Bluetooth range, with the respective personal device transmitting the broadcast signals.

In embodiments, the technology disclosed provides a solution for dynamically and accurately determining the indoor position or location for a personal device by measuring, by each of the network nodes which are within short-range communication range with the personal device, the signal strength of short-range wireless broadcast signals, e.g. Bluetooth broadcast signals, received from the personal device.

In embodiments, the controller node for the personal device may then receive, via the IP-based network, data and/or information representing or reflecting the measured signal strengths from each of the plurality of nodes within short-range communication range with the personal device and, based on the data and/or information (including signal strengths measured by the current controller node itself and/or the current connection node for the personal device if any of these nodes are within short-range wireless communication range with the personal device to receive the broadcasts), determine the current position or location for the personal device.

In certain embodiments, the controller may in addition use other obtained sensor data, e.g. sensor data obtained from the movement sensor of the personal device, e.g. an accelerometer, to determine and/or calculate the current position and/or location for the personal device.

In certain embodiments, the controller node may forward the obtained data and/or information representing or reflecting the measured signal strengths received from the network nodes within short-range communication range with the personal device, and/or other movement sensor data obtained, to a locally-installed or remote processing arrangement such as a backend system and network which determines the current position or location for the personal device.

In certain embodiments, the current position or location, e.g. room, for the personal device may at least partly be determined by a trained machine learning algorithm used by the controller node or a locally-installed or remote processing arrangement such as a backend system communicatively coupled to the controller node, since machine learning is an efficient way of evaluating complex signals in situations where large amounts of data is collected. The trained machine learning algorithm may then use at least the signal strength received from the nodes within short-range wireless communication range (including the current controller node and the current connection node for the personal device if any of these two nodes are within short-range wireless communication range with the personal device to receive the broadcasts) and/or sensor data detected by a movement sensor of the personal device to determine the current position or location for the personal device.

In aspects and embodiments, the technology disclosed relates to a method for a system comprising at least two or at least three network nodes configured to share data with the other nodes via an IP-based network and a plurality of personal devices each with Bluetooth communication capabilities, the method comprising the steps of:
a. establishing, by a connection node assigned to a personal device, a short-range wireless communication link between the connection node and the personal device, where the short-range wireless communication link is the only current communication link between the personal device and the network of nodes;
b. transmitting, from the personal device, data via the established short-range wireless communication link to the connection node; and
c. transmitting, from the personal device, short-range wireless broadcast messages, where the short-range wireless broadcast messages are transmitted while the personal device is transmitting data over and/or has the established short-range wireless communication link with the connection node assigned to the personal device.

In aspects and embodiments, the technology disclosed relates to a method for a system comprising network nodes configured to share data with the other nodes via an IP-based network and a plurality of personal devices each with Bluetooth communication capabilities, the method comprising the steps of:
a. assigning, e.g. automatically by the nodes of the system, only one single controller node among the network nodes to have the sole responsibility for a personal device;
b. assigning, by the controller node, only one single connection node for the personal device, where the controller node is sole responsible for assigning connection node for the personal device;
c. establishing, by the assigned connection node, a short-range wireless communication connection link between the connection node and the personal device, where the short-range wireless communication connection link is the only current connection link between the personal device and the network of nodes;
d. transmitting, from the personal device, data via the established short-range wireless communication connection link to the connection node; and
e. transmitting, from the personal device, short-range wireless broadcast messages, where the short-range wireless broadcast messages are transmitted while the personal device is transmitting data over the established short-range wireless communication link with the connection node.

In aspects and embodiments, the technology disclosed relates to a method for a system comprising at least two network nodes configured to share data with the other nodes via an IP-based network and a plurality of personal devices with Bluetooth communication capabilities, said method comprising:
a. assigning, e.g. by the nodes of the system using a common decision model which is known to all of the at least two network nodes, only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only currently established Bluetooth connection from the network to said personal device;
d. transmitting, from said personal device, data via said established Bluetooth connection to said only one single connection node; and
e. transmitting, from said personal device, Bluetooth broadcast signals, wherein said Bluetooth broadcast signals are transmitted while said personal device has said established Bluetooth connection with said only one single connection node.

In embodiments, the technology disclosed relates to a method for a system comprising at least two network nodes and a plurality of personal devices with Bluetooth communication capabilities, said method comprising:
a. assigning, e.g. by the nodes of the system using a common decision model which is known to all of the at least two network nodes, only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. identifying, by said personal device, at least one event associated with said personal device or the user or wearer of said personal device;
e. transmitting, from said personal device, event data representing, or adapted to be used for identifying, said at least one event via said established Bluetooth connection to said only one single connection node;
f. transmitting or forwarding, from said connection node and following or in response to receiving said event data by said connection node, event data representing, or adapted to be used for identifying, said at least one event to said controller node;
g. transmitting, from said personal device, event data representing, or adapted to be used for identifying, the same at least one event in at least one Bluetooth broadcast message, wherein said at least one Bluetooth broadcast message is transmitted concurrently or in parallel with said transmission of event data via said established Bluetooth connection and while said personal device has said established Bluetooth connection with said only one single connection node;
h. receiving, by a plurality of network nodes within Bluetooth broadcast range with said personal device, said at least one Bluetooth broadcast message containing event data representing, or adapted to be used for identifying, said at least one event; and
i. transmitting, from at least one network node within Bluetooth broadcast range with said personal device, event data representing, or adapted to be used for identifying, said at least one event to said controller node.

In embodiments, the technology disclosed relates to a method for a system comprising at least two network nodes configured to share data with the other nodes via an IP-based network and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only currently established Bluetooth connection between the at least two network nodes constituting the IP-based network and said personal device;
d. identifying, by said personal device, at least one occurred event associated with said personal device;
e. transmitting, from said personal device, event data representing said at least one occurred event via said established Bluetooth connection to said only one single connection node;
f. transmitting or forwarding, from said connection node and in response to receiving said event data by said connection node, event data representing said at least one occurred event to said controller node via said IP-based network;
g. transmitting, from said personal device, event data representing said same at least one occurred event in at least one Bluetooth broadcast message, wherein said at least one Bluetooth broadcast message is transmitted concurrently or in parallel with said transmission of event data via said established Bluetooth connection and while said personal device has said established Bluetooth connection with said only one single connection node;
h. receiving, by a plurality of network nodes within Bluetooth broadcast range with said personal device, said at least one Bluetooth broadcast message containing event data representing said same at least one occurred event; and
i. transmitting, from at least one network node within Bluetooth broadcast range with said personal device, event data representing said at least one occurred event to said controller node via said IP-based network.

In various embodiments, the concurrent or parallel transmissions of event data representing the same at least one event from the personal device via both the established Bluetooth connection and via at least one Bluetooth broadcast message are triggered by at least one event identified by the personal device.

In embodiments, the at least one event identified by the personal device and which triggers the parallel and redundant transmissions of event data may be sensor data sensed or measured by a sensor of the personal device or may be triggered by activating, by the individual or object carrying or wearing the personal device, an input means of the personal device such as the event of pressing of a button, e.g. an alarm button, on the personal device or an event identified by voice activation using a voice activation means of the personal device.

In embodiments, the sensed or measured sensor data that triggers the parallel and redundant transmissions of event data representing the at least one event may include that the personal device identifies that a sensed or measured sensor data exceeds or under-runs at least one predefined threshold value, e.g. a predefined threshold temperature value such as a predefined threshold body temperature value of the individual or object carrying or wearing the personal device.

In embodiments, the sensor which sensed or measured sensor data triggers the parallel or redundant transmissions of event data from the personal device may be a location or position detector or sensor, e.g. an RFID means, for determining the current position or location of the individual carrying or wearing the personal device. The sensed or measured sensor data that triggers the parallel and redundant transmissions of event data representing the at least one event may then include that the personal device identifies that the individual or object carrying or wearing the personal device has changed his/her location or position by receiving or exchanging, e.g. wirelessly receiving or exchanging location or position data, from/with a wireless transmitter or transceiver. The received or exchanged location or position data may then be used by the personal device to determine that the personal device is in a certain position or location, e.g. a certain room, of a facility in which a monitored individual may be residing, e.g. a nursing home, where there may be a private space for the monitored individual, including e.g. a bedroom, as well as public spaces that are shared by a plurality of monitored individuals. The parallel or redundant transmissions of event data may also be at least partly based on a timer of the personal device is indicating a certain movement pattern of the individual wearing the personal device, and/or at least partly based on a timer of the personal device is indicating that a certain predefined time period has lapsed since the individual activated an input means of the personal device, e.g. that a certain predefined time period has lapsed since the individual pressed a button of the personal device

In embodiments, the sensor which sensed or measured sensor data triggers the parallel or redundant transmissions of event data from the personal device may be a movement sensor such as an accelerometer for determining the movement speed and/or pattern of the individual carrying or wearing the personal device. The sensed or measured sensor data that triggers the parallel and redundant transmissions of event data representing the at least one event may then include that the personal device identifies that a sensed or measured sensor data exceeds or under-runs at least one predefined threshold value, e.g. a predefined threshold movement speed, a predefined threshold average movement distance per time unit and/or threshold movement pattern value of the individual or object carrying or wearing the personal device.

In embodiments, the sensor which sensed or measured sensor data triggers the parallel or redundant transmissions of event data from the personal device may be a health or medical sensor such a body temperature sensor for determining the body temperature of the wearer, or may nclude a blood pressure sensor, a heart rate sensor, a skin galvanometric level sensor etc.

In embodiments, the personal device may be a wristband and at least one sensor of the personal device may include at least one health or medical sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a temperature sensor for obtaining sensor data for determining the body temperature of the wearer, or may include a blood pressure sensor, a heart rate sensor, a skin galvanometric level sensor etc.

In embodiments, the technology disclosed relates to a method for determining the current position or location for a personal device of a system comprising at least two network nodes and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. transmitting, from said personal device, data via said established Bluetooth connection to said only one single connection node;
e. transmitting, from said personal device, data in at least one Bluetooth broadcast message, wherein said at least one Bluetooth broadcast message is transmitted concurrently or in parallel with said transmission of data via said established Bluetooth connection and while said personal device has said established Bluetooth connection with said only one single connection node;
f. receiving, by a plurality of network nodes within Bluetooth broadcast range with said personal device, said at least one Bluetooth broadcast message;
g. measuring, by said plurality of network nodes within Bluetooth broadcast range with said personal device, signal strength values for the received at least one Bluetooth broadcast message;
h. transmitting or forwarding, from the plurality of network nodes within Bluetooth broadcast range with said personal device, said measured signal strength values, or data representing or reflecting said measured signal strength values, to said controller node; and
i. determining, by said controller node, the current position or location for the personal device at least partly based on the received measured signal strength values separately received from the plurality of network nodes.

In embodiments, the technology disclosed relates to a method for determining the current position or location for a personal device of a system comprising at least two network nodes and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. transmitting, from said personal device, data via said established Bluetooth connection to said only one single connection node;
e. transmitting, from said personal device, data in at least one Bluetooth broadcast message, wherein said at least one Bluetooth broadcast message is transmitted concurrently or in parallel with said transmission of data via said established Bluetooth connection and while said personal device has said established Bluetooth connection with said only one single connection node;
f. receiving, by a plurality of network nodes within Bluetooth broadcast range with said personal device, said at least one Bluetooth broadcast message;
g. measuring, by said plurality of network nodes within Bluetooth broadcast range with said personal device, signal strength values for the received at least one Bluetooth broadcast message;
h. transmitting or forwarding, from the plurality of network nodes within Bluetooth broadcast range with said personal device, said measured signal strength values, or data representing or reflecting said measured signal strength values, to said controller node; and
j. determining, by said controller node or a locally-installed or remote processing arrangement communicatively coupled to the controller node, the current position or location for the personal device at least partly based on the received measured signal strength values separately received from the plurality of network nodes.

In embodiments, the technology disclosed relates to a method for changing connection node for a personal device of a system comprising at least two network nodes and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. transmitting, from said personal device, data via said established Bluetooth connection to said only one single connection node;
e. transmitting, from said personal device, data in at least one Bluetooth broadcast message, wherein said at least one Bluetooth broadcast message is transmitted concurrently or in parallel with said transmission of data via said established Bluetooth connection and while said personal device has said established Bluetooth connection with said only one single connection node;
f. receiving, by a plurality of network nodes within Bluetooth broadcast range with said personal device, said at least one Bluetooth broadcast message;
g. measuring, by said plurality of network nodes within Bluetooth broadcast range with said personal device, signal strength values for the received at least one Bluetooth broadcast message;
h. transmitting or forwarding, from the plurality of network nodes within Bluetooth broadcast range with said personal device, said measured signal strength values, or data representing or reflecting said measured signal strength values, to said controller node; and
k. determining, by said controller node or a locally-installed or remote processing arrangement communicatively coupled to the controller node, to change connection node, or assign a new connection node, for said personal device, wherein said change of connection node is at least partly based on the received measured signal strength values separately received from the plurality of network nodes, and wherein said new connection node is among said plurality of network nodes and is the new only one single connection node for said personal device.

In aspects and embodiments, the technology disclosed relates to a system including a network comprising at least two network nodes configured to share data with the other nodes via an IP-based network and a plurality of personal devices with Bluetooth communication capabilities, said plurality of personal devices are each assigned only one controller node among the at least two network nodes, wherein the only one controller node is responsible for assigning only one connection node among the at least two network nodes to be responsible for establishing a Bluetooth connection with the personal device the controller node is responsible for, and wherein said plurality of personal devices are each configured to be transmitting Bluetooth broadcast signals while having an established Bluetooth connection with their respectively assigned only one connection node.

In embodiments, the only one controller node assigned to the respective personal device is configured to assign and dynamically change which of the at least two network nodes is currently the only one connection node for their respective personal devices at least partly based on at least one of contents and received signal characteristics of at least one Bluetooth broadcast signal transmitted from the personal device and which is received by a plurality of other nodes than the controller node.

In embodiments, the only one controller node assigned to the respective personal device is configured to receive data via said IP-based network from a plurality of other nodes than the controller node, and wherein said data received via said IP-based network include data indicating the measured signal strength values of Bluetooth broadcast signals transmitted by the respective personal device and which are received and measured by the plurality of other nodes.

In embodiments, the only one controller node assigned to the respective personal device is further configured to change the only one connection node for the personal devices the only one controller node is responsible for at least partly based on said data indicating the measured signal strength values of Bluetooth broadcast signals transmitted by the personal device and which are received and measured by the plurality of other nodes.

In embodiments, the only one controller node assigned to the respective personal device is further configured to determine the current position of the personal device it is responsible for based on said data indicating the measured signal strength values of Bluetooth broadcast signals transmitted by the respective personal device and which are received and measured by the plurality of other nodes.

In embodiments, the system further comprises a locally-installed or remote processing arrangement such as a backend system and network configured to receive data indicating measured Bluetooth broadcast signal strength values for a personal device from the only one controller node responsible for the personal device, and wherein said locally-installed or remote processing arrangement, .e.g. backend system and network, is further configured to determine the current position for the personal device based on the received Bluetooth broadcast signal strength values for the personal device and return said determined position to the only one controller node responsible for the personal device.

In embodiments, each of said plurality of personal devices is configured to transmit Bluetooth broadcast signals including alarm data in response to a trigger event determined by the respective personal device to have occurred, and wherein said alarm data is adapted to indicate the occurrence of said trigger event to any node receiving the Bluetooth broadcast signal and is transmitted by the respective personal device while having an established Bluetooth connection with their respectively assigned only one connection node.

In embodiments, each of said plurality of personal devices is further configured to, in addition, transmit data including redundant alarm data in response to the determined occurrence of the trigger event via the established Bluetooth connection with their respectively assigned only one connection node, and wherein the respectively assigned only one connection node is configured to, in response to receiving said alarm data via said established Bluetooth connection, at least one of transmit and forward data including alarm data via said IP-based network to the respectively assigned only one controller node, where said alarm data is adapted to indicate to the only one controller node that the trigger event has occurred.

In embodiments, said system comprises at least one mobile communication device and a backend system and network configured to receive alarm data indicating the occurred trigger event from the only one controller node responsible for the personal device which determined that the trigger event occurred, and wherein said backend system and network is further configured to transmit alarm data to the at least one mobile communication device in response to receiving said alarm data from the only one controller node.

In embodiments, the local or remote processing arrangement, e.g. backend system and network, is further configured to transmit said alarm data to the at least one mobile communication device via a network separate from said IP-based network used by the at least two network nodes to exchange data.

In embodiments, said trigger event is the activation of an input means on the respective personal device.

In embodiments, said personal device is a wristband and said trigger event is the action of pressing a button on the wristband or activating a voice activation means of the personal device.

According to certain aspects and embodiments, the assignment of roles for the network nodes is associated with a specific individual personal device and is done on two levels, a control level through the assignment of a controller node and a connect level through the assignment of a connection node. The role assigned to a network node may be dynamically changed based on data obtained by the personal devices and/or the node network.

The system, e.g. monitoring system, comprising the node network of the technology disclosed is highly scalable and provides reliable and fast responses to obtained sensor data, event data and inputs from the monitored individuals, yet is robust in that each of the personal devices always has a reliable short-range wireless connection to the node network which is not disturbed or compete for short-range wireless bandwidth with other devices connected to the same node of the node network.

According to certain aspects and embodiments, the system and methods of the technology disclosed provides a scalable solution in the dynamic reconfiguration of the node networks on two levels, yet is robust and reliable in that each of the personal devices always is provided a reliable short-range wireless connection to the network. In embodiments, this is achieved by assigning a controller node to each personal device which is responsible for assigning a connection node to the personal device and dynamically changing connection node for the personal device, where the connection node is responsible for establishing a short-range wireless connection with the personal device.

According to aspects of the technology disclosed, the other network nodes shares or forwards any received or obtained data or information, e.g. status data, sensor data, event data or input data, which is received from or associated with a certain personal device to the controller node for the personal device. This allows for redundancy and, thus, safer and more accurate decision-making by the controller node, as well as provides faster responses to events, e.g. the pressing of a button on the personal device, and obtained sensor data in that all data or information associated with a certain personal device is quickly available to the one controller node having the sole responsibility for taking actions and making decisions on behalf of the personal device, e.g. fast responses in terms of making a decision to set an alert, change a health state and/or change connection node for the personal devices.

According to aspects of the technology disclosed, there is only one controller node for each personal device and the system is configured so that each of the other network nodes shares or forwards any received or obtained data or information, e.g. status data, sensor data, event data or input data, associated with a certain personal device to the controller node for the personal device. This provides for redundancy and, thus, safer and more accurate decision-making by the controller node as well as faster responses to events and obtained data in that all data or information associated with a certain personal device is received by the one controller node having the sole responsibility for making decisions and taking actions on behalf of the personal device.

In aspects, the technology disclosed proposes a system and node network for dynamically assigning roles to the nodes of the network on two levels, a control level and connect level. Each of the personal device may then be assigned only one network node, on the control level, to be controller node for the personal device and only one network node, on the connect level, to be the connection node for the personal device. The assigned controller is then responsible for dynamically assigning only one connection node for the personal device at a time. The controller node assigned to a personal device may be assigned according to a common decision model known to and used by the at least two network nodes and following the sharing or distribution of data associated with the personal device. In embodiments, the data shared between the nodes may then be used as input values to the common decision model for dynamically changing controller node for the personal device.

According to aspects of the technology disclosed, there is only one connection node for establishing a short-range wireless connection with the personal device and the system is configured so that the controller node for the personal device has the sole responsibility for changing connection node for the personal devices. This provides for a fast response yet robust and scalable solution for monitoring personal devices, e.g. wristbands, necklaces or clips carried or worn by individuals moving between locations or rooms of a facility such as patients in a nursing home. The established short-range wireless connection is then the only connection between the network and the personal device. According to this embodiment, the personal device may then still transmit short-range wireless broadcast signals to provide the other network nodes within short-range wireless communication range with status data, obtained sensor data, event data and/or input data associated with the personal device.

In certain embodiments of the technology disclosed, the system is configured so that the connection node assigned to a personal device is never the assigned controller node for the personal device.

In embodiments of the technology disclosed, the system is configured so that the connection node assigned to a personal device is never the currently assigned controller node for the personal device, yet the controller node and connection node assigned to a personal device may be dynamically changed so that a node that previously was connection node for a personal device may be the controller node for the personal device, and vice versa.

According to aspects of the technology disclosed, the wireless transmissions of data between the personal devices and the nodes of the network of nodes are thereby separated from the transmissions, e.g. wireless transmission, of data between the nodes of the node network. The data shared between the nodes of the network may then be transmitted over an IP-based network, e.g. WiFi or Ethernet, and the data transmitted from the personal devices to the node network and the data exchanged between the personal devices and the individual nodes of the network may be transmitted via short-range wireless communication, e.g. using a short-range wireless communication protocol such as Bluetooth or other protocols providing similar short-range communication range.

According to aspects of the technology disclosed, the data transmitted from the personal devices to the node network and between the personal devices and the network nodes may then contain sensor data or event data obtained and transmitted by the personal devices and then received by at least one network node within short-range wireless communication range. The transmission of data between the at last three network nodes may include at least one of the transmission of data associated with a certain personal device which is transmitted or forwarded from one of the nodes to the assigned controller node for the personal device, transmission of data which is shared between the nodes prior to assigning a node to be the controller node for a personal device and the transmission of data from the controller node to inform and/or instruct a (new) connection node which is assigned, e.g. dynamically assigned, by the controller node to be the connection node for a personal device and which is assigned to establish a short-range wireless connection with the personal device.

The technology disclosed provides a more scalable system, e.g. monitoring system, comprising a node network that does not rely on the presence of other personal devices within short-range communication range for making decisions on behalf of a personal device and, since the personal devices connected to the node network are not sharing their short-range wireless communication interface to the node network with short-range wireless connections to other personal devices, is less dependent on the currently available short-range wireless bandwidth. The technology disclosed comprising a node network where the nodes are transmitting obtained data associated with a certain personal device to the controller node responsible for the personal is therefore less dependent on the current number of other short-range wireless connections to the communication interface of the personal devices.

According to aspects, the controller node having the sole responsibility for a certain personal device is responsible for assigning and dynamically changing which of the at least two network nodes is the connection node for the personal device. The controller node may then decide to change connection node for a personal device the controller node is responsible for at least partly based on data received from the other nodes, e.g. measured signal strengths of short-range broadcast signals or messages transmitted by the personal device and which are received by any of the other nodes which are within short-range communication range with the personal device. The controller node may thereby see to that the personal device always has the best possible or available short-range wireless connection, e.g. Bluetooth connection, to the network of nodes which in turn may be communicatively coupled to a backend system. This provides for a scalable system which responds quickly to events, obtained sensor data and the movements of the individuals in that data is shared between the nodes, yet is robust in that each of the personal devices is continuously and always provided with a reliable short-range wireless connection to the network.

The dynamic assignment of connection node by the controller node by taking into account at least one of signal strengths of broadcast signals measured by the nodes within short-range wireless communcation range and the number of current short-range connections to the other network nodes also provides improved load-balancing in the network. The controller node, or a local or remote processing arrangement such as a backend system communicatively coupled to the controller node, may further use a more advanced load balancing algorithm to further improve the load-balancing in the network where the load-balancing algorithm may consider at least one of the number of personal devices each of the network nodes is controller node for, the number of short-range wireless connections in a certain location or geographical sub-area of the facility and the number of personal devices in a certain location or geographical sub-area of the facility. In aspects, the technology disclosed relates to a system comprising a node network which is self-organizing by assigning a controller node to each personal device within short-range communication range with at least one of the nodes of the network.

In embodiments, the assignment of a controller node may use a common decision model which is known to the nodes of the node network where the decision model may, or may not, use input or parameter data values obtained from the personal devices and/or other nodes of the network to determine which of the at least two nodes is going to be the controller node. The input or parameter data values may change the output from the use of the common decision model and the determining of which of the network nodes is going to be the controller node for a personal device. In some embodiments, the controller node assigned to a certain personal device may be dynamically changed at least partly based on obtained input or parameter data values.

In embodiments, the assignment, by the controller node, of a connection node may be at least partly based on signal strengths of short-range wireless broadcast signals which are measured by a plurality of network nodes within short-range communication range with the personal device transmitting the short-range wireless broadcast signals. The assignment of connection node by the controller node may further be based on or use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

In embodiments, the technology disclosed relates to a personal device, a system comprising at least two network nodes and methods in a system comprising a Self-Organizing Network (SON) and a plurality of personal device, e.g. a plurality of wristbands worn by individuals such as patients.

In aspects, the technology disclosed relates to methods for dynamically assigning roles to network nodes, a system for monitoring a plurality of personal devices and a portable personal device with short-range communication capability for communicating with network nodes.

The system, e.g. monitoring system, may comprise a memory configured to store obtained or received data associated with the individual personal devices, e.g. data detected or registered by a sensor of the individual personal device or measurement data provided by network nodes of the system. The system may further comprise a data processing unit and computer program code control logics.

In aspects and certain embodiments, the technology disclosed relates to a system including a network comprising at least two network nodes configured to transmit data to the other nodes via the network and a plurality of personal devices with short-range wireless communication capabilities for communicating with the at least two network nodes. The plurality of personal devices are each assigned only one controller node among the at least two network nodes, and the respective controller node is responsible for assigning a connection node among the at least two network nodes to establish a short-range wireless communication connection with a personal device the controller node is responsible for.

In embodiments, the system, e.g. monitoring system, is configured so that each of said plurality of personal devices is assigned only one controller node which has the sole responsibility for the personal device.

In embodiments, said system is configured so that each or at least one of the at least two network nodes may be the assigned sole controller node for a plurality of personal devices.

In embodiments, said system including the at least two network nodes is configured to use a common decision model which is known to each of the at least two nodes for determining which of the at least two nodes is going to be the controller node assigned to the respective personal device.

In embodiments, said common decision model use input values or parameters which are continuously and dynamically changed at least partly based on data distributed and shared between the network nodes, thereby allowing for the determining of which of the nodes among the at least two nodes is going to be the controller node for the personal device to be dynamically changed at least partly on data distributed and shared between the network nodes. The network for distributing and sharing data between the network nodes is typically an IP-based network providing for improved scalability, e.g. a WiFi or Ethernet.

In embodiments, said system is configured so that each assigned network node to be connection node for a personal device only has one established short-range wireless communication connection with the node network at a time.

In embodiments, said plurality of personal devices are each configured to be transmitting short-range wireless broadcast signals while having an established short-range wireless communication connection with their respective connection node.

In embodiments, the only one controller node assigned to the respective personal device is configured to assign and dynamically change which of the at least two network nodes is currently the only one connection node for the personal device at least partly based on at least one of contents and received signal strengths of at least one short-range wireless broadcast signal transmitted from the personal device and which is received by a plurality of other nodes than the controller node.

In embodiments, the only one controller node assigned to the respective personal device is configured to receive data from a plurality of other nodes than the controller node via an IP-based network connecting the at least two network nodes, and wherein said data received via said IP-based network include data indicating the measured signal strength values of short-range wireless broadcast signals transmitted by the respective personal device and which are received and measured by the plurality of other nodes.

In embodiments, the only one controller node assigned to the respective personal device is further configured to determine a position or location for the personal device and/or change the only one connection node for the personal devices the only one controller node is responsible for at least partly based on said data indicating the measured signal strength values of short-range wireless broadcast signals transmitted by the respective personal device and which are received and measured by the plurality of other nodes and, in addition, at least partly based on signal strengths measured by the current controller node and the current connection node for the personal device if any of these nodes are within short-range wireless communication range with the personal device to receive the broadcasts.

In embodiments, the network comprises at least two network nodes configured to transmit data to the other nodes via the network is an IP-based network separated from and using a different communication protocol than used for the short-range wireless connections established between the personal devices and the network nodes. The short-range wireless capabilities of the plurality of personal devices for communicating with the at least two network nodes may then typically be Bluetooth communication capabilities, or similar short-range wireless capabilities.

In embodiments, said plurality of personal devices may include a plurality of wristbands, necklaces or clips, each may be provided with a sensor or an input means and worn by an individual such a patient of a nursing home.

In aspects, the technology disclosed relates to a method in a system, e.g. a monitoring system, including a network comprising at least two network nodes and a plurality of personal devices with short-range wireless communication capabilities, said method comprising:
a. assigning only one node among the at least two network nodes to be the controller node having the sole responsibility for a particular personal device;
b. assigning, by said controller node, a connection node for establishing a short-range wireless communication connection between said connection node and said personal device;
c. establishing, by said assigned connection node, a short-range wireless communication connection with said personal device;
d. transmitting, from said personal device and via said established short-range wireless communication connection, a message to said connection node;
e. receiving, by said connection node, said message, wherein said message contains data including at least one of status data, sensor data and event data associated with said personal device;
f. transmitting, from the assigned connection node to the sole controller node for said personal device, said received message or a message including data corresponding to, or at least partly based on, said at least one of status data, sensor data and event data associated with said personal device; and
g. transmitting, from said controller node to at least one of at least one of the other at least two network nodes and a backend system, a message containing data adapted to be used for at least one of controlling and monitoring the personal device, wherein said message is at least partly based on said data corresponding to, or at least partly based on, said at least one of status data, sensor data and event data associated with said personal device.

In embodiments, the short-range wireless communication connection is the only currently established communication connection between the network nodes and said personal device.

In embodiments, each of the plurality of personal devices is assigned only one of at least three network nodes to be the controller node for the respective personal device.

In embodiments, each of the plurality of personal devices is assigned, by their respective controller node, only one of at least three network nodes to be the connection node for the personal device.

In embodiments, each of the at least three network nodes has no more than one established short-range wireless connection with a personal device at a time.

In embodiments, the above method further comprises distributing or sharing data between at least three network nodes via an IP-based network not using the same short-range wireless communication protocols used by the personal devices for communicating with the node network. The distributing or sharing of data between the nodes is preceding the assignment of the controller node, and the data distributed or shared between at least three network nodes is further used for determining which of at least three nodes is going to be the controller node for the personal device.

In embodiments, the distributing or sharing of data between the nodes is preceding the assignment of the controller node, and the data distributed or shared between at least three network nodes is used for determining which of the at least three nodes is going to be the controller node for the personal device.

In embodiments, the method further comprises determining which of at least three nodes is going to be the controller node for the personal device by using a common decision model which is known to each of the at least three nodes.

In embodiments, the common decision model use input values or parameters which are continuously and dynamically changed at least partly based on data distributed and shared between the network nodes, thereby allowing for the determining of which of the nodes among at least three nodes is going to be the controller node for the personal device to be dynamically changed at least partly on data distributed and shared between the network nodes.

In embodiments, the method further comprises forwarding, by at least a plurality of nodes within short-range wireless communication range with the personal device, at least a portion of the contents of said short-range wireless broadcast signals to the other network nodes. The at least a portion of the contents of said broadcast signals may then be used by the network of nodes as input values to the for determining which of at least three nodes is going to be the controller node for the personal device.

In embodiments, the method further comprises forwarding, by at least a plurality nodes within short-range wireless communication range with the personal device, at least a portion of the contents of said short-range wireless broadcast signals to the controller node. The contents of said broadcast signals may then be adapted to be used by the controller node, or by a local or remote processing arrangement, for determining the position or location of the personal device for determining which of at least three nodes is to be assigned connection node for the personal device.

The at least one sensor of the personal device may include an accelerometer adapted to detect the movements of the portable personal device, e.g. the acceleration and movement pattern of the portable personal device.

In certain embodiments, the technology disclosed relates to methods and a system for monitoring an individual within predetermined facilities by identifying an event associated with a personal device carried or worn by the individual, e.g. with the purpose of determining whether there is a need for setting an alert. The system may comprise at least three network nodes and a plurality of personal devices each carried or worn by the respective monitored individual, where each of the plurality of personal devices comprises at least one sensor and has short-range communication capability, e.g. Bluetooth communication capability, with which the respective personal device is arranged to communicate with the network nodes. The personal device may also comprise a storage means and at least one processing device.

In embodiments, the personal device may be a wristband and the at least one sensor of the personal device may comprise at least one medical sensor, or health sensor, adapted for obtaining sensor data for determining the medical and/or physical condition of the wearer of the wristband, e.g. a medical sensor configured to sense a characteristic of the user and generate a sensor output signal.

In embodiments, the personal device may be a wristband and the at least one sensor of the personal device may comprise at least one health sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a body temperature sensor for obtaining sensor data for determining the body temperature of the wearer, or may include a blood pressure sensor, a heart rate sensor, a skin galvanometric level sensor etc.

In embodiments, the at least one processing device of the personal device may be arranged to receive sensor data from the at least one sensor in the personal device and determine whether the received sensor data indicates an alert state, e.g. a specific alert state among a plurality of predetermined alert states.

In certain embodiments, the alert states may comprise at least a FALL alert state to be used if a probable fall has been detected for the monitored individual and an OUT OF BED alert state to be used if it has been detected that the monitored individual is probably getting out of bed.

According to certain aspects of the technology disclosed, personal devices communicate with nodes in the node network using Bluetooth, e.g. Bluetooth broadcasts or via an established Bluetooth connection, whereas the communication between the network nodes takes place using the IP addresses of the nodes (e.g. via Wi-Fi). The network of nodes may then be self-organizing in that the nodes of the network continuously and/or automatically assigns roles to the individual nodes, where the assignment of a role is preceded by, and at least partly based on, data distributed and/or shared between the network nodes. The distributed and/or shared data is obtained by the other network nodes and is associated with the personal device to which the assignment of a role concerns.

The personal devices may broadcast their own universal unique identifiers (UUIDs) and seek to establish connections with Bluetooth devices within its Bluetooth communication range. The network nodes may be configured to store the UUIDs of all personal devices detected in their memories. The connection node assigned, by the controller node, to a certain personal device may then establish a Bluetooth connection with the personal device by transmitting a connection request via Bluetooth to the personal device where the connection request may include the unique identifier (UUID) of the personal device.

According to aspects, the network nodes may be self-organizing in that the assignment of the one controller node having the sole responsibility for a specific personal device is a distributed decision which is preceded by at least one of distributing and sharing data between the network nodes using the IP-based network and by using a predetermined and common decision model with dynamic input values/parameters which are changed with the data distributed and shared between the network nodes.

According to embodiments, the network nodes may be self-organizing in that the decision to change controller node for a specific personal device is a distributed decision which is preceded by at least one of distributing and sharing of data between the network nodes using the IP-based network and by using a predetermined and common decision model with dynamic input values/parameters which are changed with the data distributed and shared between the network nodes.

According to aspects of the technology disclosed, personal devices transmits data to the nodes in the node network using Bluetooth, e.g. via Bluetooth broadcasts and an established Bluetooth connection with one of the nodes, and the transmission of data, e.g. data associated with the individual personal device, takes place using their IP addresses, e.g. data received from a certain personal device or associated with a certain personal device may be shared between network nodes via the IP-based network connecting the network nodes.

According to aspects, the personal devices are not part of the IP-based network connecting the but are only configured to transmit data and/or communicate with the network nodes via Bluetooth, e.g. using an established Bluetooth connection with one of the nodes and transmitting Bluetooth broadcast signals which may be received (and measured) by any network node within Bluetooth communication range from the personal device.

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device;
b. assigning, by said assigned controller node, a connection node for the same personal device, where controller node is responsible for assigning said connection node to said personal device;
c. establishing, by the assigned connection node, a Bluetooth connection between the connection node and the personal device; and
d. transmitting, from the personal device, data via the established Bluetooth connection to the connection node;

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device, wherein said step of assigning the controller node is preceded by the sharing of data between the at least three nodes via the IP-based network;
b. assigning, by said assigned controller node, a connection node for the same personal device, wherein said controller node is responsible for assigning said connection node to said personal device;
c. establishing, by the assigned connection node, a Bluetooth connection between the connection node and the personal device; and
d. transmitting, from the personal device, data via the established Bluetooth connection to the connection node;

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device, wherein said only one controller node for said personal device is assigned by at least one of sharing data between the at least three network node and using a predetermined and common decision model with dynamic input values/parameters;
b. receiving, by a plurality of the at least three network nodes, Bluetooth broadcast data from said personal device;
c. forwarding, by said plurality of the at least three network nodes via the IP-based network and to said assigned only one controller node for said personal device, at least one of said received Bluetooth broadcast data, data or information corresponding to said received Bluetooth broadcast data and measured signal strength values for said received Bluetooth broadcast data;
d. assigning, by said assigned only one controller node, a connection node for said personal device, wherein said controller node is sole responsible for assigning connection node to said personal device, and wherein said assigning of connection node by said controller node is at least partly based on said at least one of said received Bluetooth broadcast data, data or information corresponding to said received Bluetooth broadcast data and measured signal strength values for said received Bluetooth broadcast data;
e. establishing, by said assigned only one connection node, a Bluetooth connection between the connection node and said personal device; and
f. transmitting, from said personal device, data via the established Bluetooth connection to the connection node;

In embodiments, the above method includes that the at least three network nodes (may be e.g. at least 5, 10, 20 or more network nodes) share data or information between each other via the IP-based network, e.g. an Ethernet or Wireless Fidelity (WiFi) network. The advantages of having the network nodes sharing data via an IP-based network whereas the personal devices connect to the network via Bluetooth connections include that the data may be distributed between.

In embodiments, the assignment of controller node for a personal device may then be performed by distributing data or information received from or associated with the personal device to all of the at least three network nodes prior to deciding which of the at least three network nodes is going to be the controller node for the personal device. According to embodiments, the (distributed) decision between the at least three network nodes as to which of the at least three network nodes will be the controller node, e.g. new controller node, for the personal device is made according to a predetermined scoring system which is known to all of the at least three network nodes, where the scoring system may use dynamic variable input values. As an example, the at least three network nodes, e.g. constituting a Self-Organizing Network, may use a predetermined decision model, e.g. distributed consensus model. In various embodiments, the predetermined decision model may define a static hierarchy for the nodes or may have dynamic input values/parameters, where the input values/parameters may include, for example, data related to load balancing between different parts of the network and an assessment of the current traffic load distribution between the at least three nodes of the IP-based network, obtained (current) position data and/or signal strength values for personal devices broadcasting and/or having an established Bluetooth connection to one of the at least three network nodes, the number of personal devices each of the at least three network nodes is currently controller node for and the number of personal devices each of the at least three network nodes is currently connection node for.

In embodiments, the above method includes that the plurality of personal devices (may be e.g. at least 5, 10, 20, 50 or more personal devices) all have Bluetooth communication capabilities and all transmit Bluetooth broadcast messages/signals which may be received by any of the at least three network nodes (may be e.g. at least 5, 10, or more network nodes) within Bluetooth communication range.

In embodiments, the above method includes that said Bluetooth connection is the only currently established Bluetooth connection from the network to said personal device and the connection node is the only network node having an established connection link with the personal device.

In embodiments, the technology disclosed relates to a method for a system including a network, e.g. a Self-Organizing Network (SON), comprising at least three network nodes configured to share data with the other nodes via an IP-based network and a plurality of personal devices with Bluetooth communication capabilities, the method comprising:
a. assigning only one controller node among the at least three network nodes to have the sole responsibility for a personal device among said plurality of personal devices;
b. assigning, by said assigned controller node, only one connection node for said personal device, wherein said controller node is sole responsible for assigning said only one connection node to said personal device;
c. establishing, by said assigned only one connection node, a Bluetooth connection between said only one connection node and said personal device, wherein said Bluetooth connection is the only currently established Bluetooth connection from the network to said personal device; and
d. transmitting, from said personal device, data via said established Bluetooth connection to said only one connection node;

In embodiments, the technology disclosed relates to a method for a system including a network, e.g. a Self-Organizing Network (SON), comprising at least three network nodes configured to share data with the other nodes via an IP-based network, e.g. an Ethernet or WiFi network, and a plurality of personal devices with Bluetooth communication capabilities, the method comprising:
a. assigning only one controller node among the at least three network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node; and
e. forwarding, by the connection node and via the IP-based network, the data received from the personal device to the assigned controller node for the personal device.

In embodiments, the technology disclosed relates to a method for assigning a connection node for a personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning one of the at least three network nodes to be the controller node with sole responsibility for a personal device;
b. transmitting, by the personal device not having an established connection with any of the at least three network nodes, Bluetooth broadcast messages/signals;
c. receiving, by a plurality of the at least three network nodes within Bluetooth communication range, at least one Bluetooth broadcast message/signal from the personal device, where the at least one Bluetooth broadcast message/signal contains unique identification data for the personal device;
d. forwarding, from said plurality of nodes to the assigned controller node via the IP-based network, position data for the personal device and/or measured signal strength values for the personal device, wherein the position data and/or measured signal strength values are obtained through said plurality of nodes receiving said at least one Bluetooth broadcast message/signal;
e. determining, by the node assigned to be the controller node with sole responsibility for the personal device, to assign only one node among the at least three network nodes to be the sole connection node for the personal device, wherein the determining by the controller node of which node will be the connection node for the personal device is at least partly based said position data and/or measured signal strength values obtained through said plurality of nodes receiving said at least one Bluetooth broadcast message/signal; and
f. establishing, by the node determined by the controller node to be connection node for the personal device, a Bluetooth connection with the personal device.

In embodiments, the technology disclosed relates to a method for changing the connection node for establishing a Bluetooth connection with a certain personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning only one controller node among the at least three network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node;
e. forwarding, by the connection node and via the IP-based network, the data received from the personal device (via the established Bluetooth connection) to the assigned controller node for the personal device;
f. transmitting, by said personal device, Bluetooth broadcast messages/signals, where the Bluetooth broadcast messages/signals are received by any of the at least three node which is within Bluetooth communication range; and
g. determining, by said controller node the personal device, a position and/or location for the personal device, where the determining of the position and/or location is at least partly based on measured signal strength values for Bluetooth broadcast messages/signals received by other nodes than the controller node and the current connection node.

In aspects, the technology disclosed relates to system comprising at least three network nodes and a plurality of personal devices, where each of the personal devices is assigned/having only one controller node and only one connection node at a time. The controller node for a personal device may then be assigned by sharing data between the nodes of the network and using a predetermined decision model, e.g. a distributed consensus model which is known to all of the at least three network nodes. In embodiments, the predetermined decision model may have dynamic input values/parameters, where the input values/parameters may include, for example, data related to load balancing between different parts of the network and an assessment of the current traffic load distribution between the at least three nodes of the IP-based network.

In aspects, the technology disclosed relates to a method in a system, e.g. a monitoring system, including a network comprising at least three network nodes configured to share data with the other nodes via an IP-based network and a plurality of personal devices with short-range wireless communication capabilities, the method comprising:
a. assigning only one controller node among the at least three network nodes to have the sole responsibility for a personal device;
b. assigning, by said controller node, only one connection node for said personal device, wherein said controller node is sole responsible for assigning said only one connection node to said personal device;
c. establishing, by said assigned only one connection node, a short-range wireless communication connection between said only one connection node and said personal device, wherein said short-range wireless communication connection is the only currently established connection from the network to said personal device;
d. transmitting, from said personal device, data via said established short-range wireless communication connection to said only one connection node; and
e. transmitting, from said personal device, short-range wireless broadcast signals, wherein said short-range wireless broadcast signals are transmitted while said personal device has said established Bluetooth connection with said only one connection node.

In embodiments, the method according to the technology disclosed is comprising:
a. receiving, by a plurality of nodes other than the only one controller node, said Bluetooth broadcast signals;
b. measuring, by each of said plurality of nodes, signal strengths values of said received Bluetooth broadcast signals;
c. transmitting, from each of said plurality of nodes and in response to receiving said Bluetooth broadcast signals, data to said only one controller node via said IP-based network, wherein said data is adapted to indicate said measured signal strengths values of said Bluetooth broadcast signals;
d. receiving, by said only one controller node and via said IP-based network, said transmitted data indicating said measured signal strengths values; and
e. determining, by said only one controller node, to change the only one connection node for said personal device at least partly based on said received data indicating said measured broadcast signal strengths values, wherein said new only one connection node for said personal device is either the assigned only one controller node or one or one of the plurality of nodes other than the controller node and is sole responsible for establishing a new Bluetooth connection to said personal device, wherein said new Bluetooth connection will be the only currently established Bluetooth connection from the network to said personal device.

In embodiments, the method according to the technology disclosed is comprising:
a. determining, by said only one controller node, the current position of said personal device based on said received data indicating said Bluetooth broadcast signal strengths values measured by said plurality of nodes.

In embodiments, the method according to the technology disclosed is comprising:
a. determining, by said personal device, that a trigger event has occurred, wherein said
b. wherein each of said plurality of personal devices is configured to transmit Bluetooth broadcast signals including alarm data in response to a trigger event determined by the respective personal device to have occurred, and wherein said alarm data is adapted to indicate the occurrence of said trigger event to any node receiving the Bluetooth broadcast signal and is transmitted by the respective personal device while having an established Bluetooth connection with their respectively assigned only one connection node.

In embodiments, the system comprises at least one mobile communication device and a backend system and network configured to receive alarm data indicating the occurred trigger event from the only one controller node responsible for the personal device which determined that the trigger event occurred, and wherein said backend system and network is further configured to transmit alarm data to the at least one mobile communication device in response to receiving said alarm data from the only one controller node.

In embodiments, the backend system and network is further configured to transmit said alarm data to at least one mobile device, e.g. at least one mobile device of a staff member of the facility, via a network separate from the IP-based network used by the at least three network nodes to exchange data.

In embodiments, the trigger event is the activation of an input means on the respective personal device.

In embodiments, the personal device is a wristband and said trigger event is the action of pressing a button on the wristband or activating a voice activation means of the personal device.

In embodiments, the only one controller node assigned to the respective personal device is configured to receive data via the network, e.g. an IP-based network, from a plurality of other nodes than the controller node. The data received via the IP-based network may then include data indicating the measured signal strength values of Bluetooth broadcast signals transmitted by the respective personal device and which are received and measured by the respective node of the plurality of other nodes.

In embodiments, the only one controller node assigned to the respective personal device is further configured to determine the current position and/or location of the personal device it is responsible for based on said data indicating the measured signal strength values of Bluetooth broadcast signals transmitted by the respective personal device and which are received and measured by the plurality of other nodes.

In embodiments, the system, e.g. monitoring system, further comprises a local or remote processing arrangement such as a backend system and network configured to receive data indicating measured Bluetooth broadcast signal strength values for a personal device from the only one controller node responsible for the personal device.

The backend system and network may then be configured to determine the current position for the personal device based on the received Bluetooth broadcast signal strength values for the personal device and return the determined position to the only one controller node responsible for the personal device.

In embodiments, each of said plurality of personal devices is configured to transmit Bluetooth broadcast signals including alarm data in response to a trigger event determined by the respective personal device to have occurred, wherein the alarm data is adapted to indicate the occurrence of said trigger event to any node within short-range wireless communication range receiving the Bluetooth broadcast signal and is transmitted by the respective personal device while having an established Bluetooth connection with their respectively assigned only one connection node.

In embodiments, each of said plurality of personal devices is further configured to, in addition, transmit data including redundant alarm data in response to the determined occurrence of the trigger event via the established Bluetooth connection with their respectively assigned only one connection node. The respectively assigned only one connection node may then be configured to, in response to receiving the alarm data via said established Bluetooth connection, at least one of transmit and forward data including alarm data via said network, e.g. IP-based network, to the respectively assigned only one controller node, where the alarm data is adapted to indicate to the only one controller node that the trigger event has occurred.

In embodiments, the system comprises at least one mobile communication device and a backend system and network configured to receive alarm data indicating the occurred trigger event from the only one controller node responsible for the personal device which determined that the trigger event occurred. The backend system may then be configured to transmit alarm data to the at least one mobile communication device of an individual such as a staff member of the facility in response to receiving the alarm data from the only one controller node.

In embodiments, the backend system and network is further configured to transmit the alarm data to the at least one mobile communication device via a network separate from the network, e.g. IP-based network, used by the at least three network nodes to exchange data.

### BRIEF DESCRIPTION OF DRAWINGS

Preferred embodiments of a system according to the technology disclosed will be described more in detail below with reference to the accompanying drawings wherein:

FIG. 1 schematically shows an example of facilities where a system comprising a number of network nodes have been installed, in accordance with one or more embodiments described herein.

FIG. 2 is a schematic illustration of a system for monitoring an individual within predetermined facilities.

FIG. 3 is an example flow diagram of a method for monitoring an individual within predetermined facilities, in accordance with one or more embodiments described herein.

FIG. 4 schematically illustrates a method for monitoring an individual within predetermined facilities, in accordance with one or more embodiments described herein.

FIG. 5 schematically illustrates a method for monitoring an individual within predetermined facilities, in accordance with one or more embodiments described herein.

FIG. 6 schematically illustrates a method for monitoring an individual within predetermined facilities, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

In the drawings, similar details are denoted with the same reference number throughout the different embodiments. In the various embodiments of the system, e.g. monitoring system, according to the technology disclosed, the different subsystems are denoted. The “boxes”/subsystems shown in the drawings are by way of example only and can within the scope of the technology disclosed be arranged in any other way or combination.

The systems and ad-hoc networks known in the art, e.g. piconets or SON, does not disclose a node network where nodes share data or information which each other prior to the dynamic assignment of roles to the nodes.

In aspects and embodiments, the technology disclosed relates to methods, a system comprising a self-organizing network (SON) of nodes and at least one wearable device each configured to transmit data associated with the respective wearable device both via an established short-range wireless connection links with one of the network nodes of the node network and via short-range wireless broadcasts which may be received by any of the network nodes that are within short-range wireless communication range with the wearable device.

In aspects and embodiments, the technology disclosed also relates to a system and methods for transmitting data associated with a personal device worn by an individual both via an established short-range wireless connection link between the personal device and a network node and via short-range wireless broadcasts from the personal device.

In aspects and embodiments, the personal device is adapted to be worn by an individual and is configured to transmit short-range wireless broadcast signals while a short-range wireless connection between the wearable device and a node of the network of nodes is active.

In embodiments, each of the at least one wearable device is assigned one of the at least one hub node to have the controller node role for the wearable device and be configured for dynamically determining and changing which network node is currently having a short-range wireless communication link established with the wearable device.

In embodiments, the at least one of input data and sensor data obtained by the wearable device is transmitted to the backend system by the one hub node assigned by the SON to have the controller node role for the wearable device, wherein the controller node is responsible for determining and dynamically changing which of the nodes of the SON is currently having an established short-range wireless connection with the wearable device.

In embodiments, the method includes that each of the at least one wearable device is assigned only one of the at least one hub node to have the controller node role for the wearable device and be configured for dynamically determining and changing which node is currently having a short-range wireless communication link established with the wearable device.

In aspects, technology disclosed relates to methods and a system comprising at least two network nodes and at least one personal devices, or wearable devices, each configured to transmit at least one short-range wireless broadcast signal which is received by any of the at least two network nodes within short-range wireless communication range with the respective personal device. The transmission of the at least one short-range wireless broadcast signal is performed while an established communication connection or link between the wearable device and one of the network nodes which is the assigned connection node for the respective personal device is active. The transmission of broadcast signals may be performed in parallel with or while the personal device is transmitting messages via the established short-range wireless communication link.

In aspects, technology disclosed relates to methods and a system comprising at least three network nodes and a plurality of personal devices, or wearable devices, each configured to continuously, e.g. periodically, transmit short-range wireless broadcast signals which are received by any of the at least three network nodes within short-range wireless communication range with the respective personal device. The transmissions of the short-range wireless broadcast signals may then be performed while an established communication link between the personal device and one of the network nodes which is the assigned connection node for the respective personal device is active. The transmission of the short-range wireless broadcast signals, e.g. Bluetooth/BLE signals, may be performed in parallel with or while the personal device is transmitting messages via the established short-range wireless connection or communication link, e.g. Bluetooth/BLE communication link.

In embodiments, the personal devices are adapted to be worn by individuals and are configured to transmit short-range wireless broadcast signals while a short-range wireless connection between the personal device and a node of the network of nodes is active. The hub node assigned to have the controller node role for a wearable device is configured to change which of the network nodes is having a short-range wireless communication link the position or location of the individual and take actions on behalf of the individual wearing the personal device and in response to received data contained in broadcast signals forwarded by other network nodes, including other hub nodes and satellite nodes, and/or status data, sensor data and/or event data received and forwarded to the controller node by the current connection node having a short-range wireless communication link with the wearable device.

According to aspects and certain embodiments of the technology disclosed, data or information contained in short-range wireless broadcast message received by the connection node, e.g. a satellite node, via the established short-range wireless communication link is forwarded via an IP-based network to the assigned controller node for the wearable device. The controller node, which has sole responsibility for the personal device, may be further configured to use input data and/or sensor data obtained by the wearable device which is forwarded from the currently assigned connection node and/or input data and/or sensor data obtained by the wearable device which is received from the other nodes to take actions or make decisions on behalf of the wearable device, e.g. to change connection node for the wearable device at least partly based on the received and obtained input data and/or sensor data.

In aspects, the technology disclosed relates to a system including a network comprising at least two network nodes and a plurality of personal devices with short-range wireless communication capabilities. The system is configured to assign a connection node among the at least two network nodes to each of the plurality of personal devices. The connection node is responsible for establishing a short-range wireless communication link with the personal device it is assigned connection node for.

In aspects, the technology disclosed relates to a system including a network comprising at least three network nodes and a plurality of personal devices with short-range wireless communication capabilities. The system is configured to assign a connection node among the at least three network nodes to each of the plurality of personal devices. The connection node is responsible for establishing a short-range wireless communication link with the personal device it is assigned connection node for.

According to the technology disclosed, each of the plurality of personal devices is configured to transmit short-range wireless broadcast signals while their respective established short-range wireless connection with their respectively assigned connection node is active.

In embodiments, the system is configured to assign a controller node for the respective of the plurality of personal device, and wherein each controller node for the respective personal device is configured to assign a connection node for the personal device and to receive, from the other network nodes, status data, sensor data and/or event data associated with the personal device it is responsible for.

In embodiments, the IP-based network connecting the hub nodes is a separate network and is configured to use a different communication protocol from the communication protocol used for establishing the short-range wireless communication links between the at least one wearable device and the network nodes, e.g. satellite nodes. The short-range wireless communication links for connecting the wearable devices to one of the network nodes, either a satellite node or a hub node, may be Bluetooth communication links, e.g. BLE links.

In embodiments, the hub nodes are configured to share data with the other nodes via an IP-based network connecting the hub nodes, e.g. all of the hub nodes of the SON may be wirelessly connected via WiFi connections or via Ethernet connections, thereby connecting the hub nodes so that they continuously can share their obtained input data and/or sensor data with the other hub nodes via distribution between the hub nodes.

In embodiments, the hub nodes are configured to share data with the other nodes via established short-range wireless communication link, e.g. Bluetooth/BLE links, thereby connecting the hub nodes so that they continuously can share their obtained input data and/or sensor data with the other hub nodes via distribution between the hub nodes.

In embodiments, the established short-range wireless communication link between the connection node and the personal device is a bidirectional short-range wireless communication link.

In embodiments, the technology disclosed provides a fast response yet robust solution for acting on an identified event by parallel and/or redundant data transmission of event data representing or reflecting the event via both an established short-range wireless connection with one of the nodes and via short-range wireless broadcast messages from the personal device to the nodes which are within short-range wireless communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location for a personal device without any use of high energy-consuming GPS in the personal devices or beacons, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

In certain embodiments, the current position or location for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically changing the connection node for a personal device, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine to change connection node for the personal device.

In various embodiments, the controller node may forward the obtained data and/or information representing or reflecting the measured signal strengths from each of the plurality of other nodes, and/or other movement sensor data obtained, to a backend network which determines that the connection node for the personal device should be changed.

In various embodiments, the controller node may in addition use other obtained sensor data, e.g. sensor data from a movement sensor of the personal device, e.g. an accelerometer, to determine that the connection node for the personal device should be changed.

In certain embodiments, the change of connection node for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine that the connection node for the personal device should be changed.

The systems and networks, e.g. piconets or SON, known in the art further does not disclose a system, e.g. monitoring system, comprising a node network and personal devices with broadcast capabilities which is self-organizing and scalable by dynamically assigning roles to the individual nodes, e.g. continuously and automatically assigning roles to the nodes, where each assignment of a role to a node relates to managing and control of only one of the personal devices. In particular, the systems and network solutions known in the art are silent to dynamically assigning and changing roles to the nodes on two levels, a control level where the node has the sole responsibility for a personal device and a connection level where the node is responsible for establishing a connection or link with the personal device.

The ad-hoc networks known in the art does not disclose or describe a node network where the nodes communicate internally via their IP addresses, but where the communication between personal devices and the network nodes takes place via a short-range wireless communication such as Bluetooth. This gives a much higher bandwidth capacity for the communication between the personal devices and the network nodes. This also enables the node network to be scalable and more distributed, since communication between network nodes can take place via the IP-based network connecting the nodes.

To address the identified problems with the prior art, the technology disclosed proposes a system, e.g. monitoring system, and node network for dynamically assigning roles to the nodes of the network on two levels, a control level and connect level.

The technology disclosed introduces a node network where the nodes communicate internally via their IP addresses, but where the communication between personal devices and nodes takes place via short-range wireless communication such as Bluetooth. This gives a better load balancing and much higher bandwidth capacity for the communication between the personal devices and the nodes. This also enables the node network to be more scalable and distributed, since communication between nodes can take place via an IP-based network.

The system, e.g. monitoring system, comprising the node network of the technology disclosed is highly scalable and provides for safe and fast responses to events, obtained data and the movements of the individuals, yet is robust in that each of the personal devices always has a reliable short-range wireless connection to the node network which is not disturbed or compete for short-range communication bandwidth with other devices connected to the same node of the node network.

In aspects, the technology disclosed allows for faster and more accurate decision-making and improved load balancing within a network by providing methods and a system comprising personal devices and a network of nodes for dynamically assigning one controller node and one connection node for each personal device. In embodiments, the controller node for a personal device is dynamically assigned or changed based on a common decision model known to the network nodes and which may contain input values or parameters. The input values or parameters may be changed with the data distributed and shared between the network nodes. In certain embodiments of the technology disclosed, the consensus model as such with its input values or parameters may also be dynamically adjusted and changed over time.

In embodiments, the technology disclosed relates to a system comprising a node network which is self-organization by including a dynamic assignment of a node as uploader node to the internet or backend system.

In certain embodiments, the technology disclosed relates to a system comprising a node network which is self-organization by assigning a “stand-by” node to take over from the assigned node, e.g. a stand-by node which is ready to take over from an assigned controller node, an assigned connection node and/or an assigned uploader node.

In certain embodiments, the assignment of a stand-by controller node may use the same common decision model as used for the assignment of the controller node where the decision model may, or may not, use input or parameter data values obtained from the personal devices and/or nodes of the network to determine which of the at least three nodes is going to be the stand-by node. The input or parameter data values may change the determining of which of the network nodes is going to be the stand-by controller node for the personal device.

In certain embodiments, the assignment, by the controller node, of a stand-by connection node to take over from the assigned connection node may be at least partly based on signal strengths of short-range wireless broadcast signals which are measured by a plurality of network nodes within short-range communication range with the personal device transmitting the short-range wireless broadcast signals. The assignment of stand-by connection node by the controller node may further use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

The faster and more accurate decision-making is at least partly achieved by the method of assigning, for each personal device, one controller node with sole responsibility for the personal device, where the controller node is provided with all data or information associated with the personal device the controller node is responsible for and which is received, obtained and/or measured by the network nodes.

The faster and more accurate decision-making may be further achieved by the controller node making all decisions for the personal device, e.g. the decision to set an alert or transmit alarm data or the assignment of connection node with which the personal device is establishing a short-range wireless communication link such as a Bluetooth connection. The connection node may then have an established connection with the personal device as long as the controller node considers it appropriate, e.g. as long as the signal strength measured by the connection node is above a certain threshold value and/or a signal strength measured by the connection node is higher than signal strengths measured by the other nodes of the network.

Improved load-balancing is achieved by having the controller node with sole responsibility for a personal device dynamically assigning and changing connection node for the personal device at least partly based on load-balancing factors such as the current number of established short-range wireless connections for a node, e.g. as compared to the number of connections for other nodes, to thereby distribute the amount of short-range wireless data transmitted locally.

In certain aspects, the system and self-organizing network of the technology disclosed separating the data traffic in the IP-based network traffic from the short-range wireless communication performed by the personal devices provides an improved solution over ad-hoc networks and many state of the art self-organizing networks in that the transmission of data between the nodes used for configuring and self-organizing the network nodes, e.g. data used for assigning different roles to the nodes, is performed via the IP-based network and not via the short-range wireless protocol the personal device are using for providing the network nodes with data, thereby does not load, affect or interfere with the short-range wireless radio traffic, e.g. the Bluetooth radio traffic.

Benefits of the technology disclosed include that the system, self-organizing network and methods provide each of the personal devices with improved short-range wireless connections by continuously and dynamically changing which of the network nodes is the sole connection node having the only short-range wireless connection, e.g. Bluetooth connection, to a personal device at least partly based on measured signal strengths for the currently established short-range wireless connection and/or short-range signal strengths measured by the individual nodes of the network.

Further benefits of the technology disclosed include that the controller node is provided with all data associated with the personal device it is responsible for in that the all data or information associated with the personal device, or data or information representing or reflecting the obtained data or information, is forwarded from the other network nodes to the controller node, including the data received and measured by the currently assigned connection node for the personal device.

In embodiments, the hub node having the controller node role for a wearable device is configured to receive at least one of input data from a user input means and sensor data obtained by a sensor of the wearable devices and continuously share received input data and/or sensor data with other hub nodes of the SON so that hub nodes having the controller node role for the wearable device receives the input data and/or sensor data and, at least partly based on the input data and/or sensor data, at least one of transmit control data to the wearable device instructing the wearable device to activate a sensor.

In embodiments, the hub node having the controller node role for a wearable device is configured to, in response to receiving downlink data from the backend system or received or obtained sensor data and/or input data which indicates at least one of an alert state, a changed physical condition, a changed health state, a changed physical orientation of the person wearing the wearable device and that a call should be established with the wearable device: transmit control data to the wearable device instructing the wearable device to activate the sensor of the wearable device.

In embodiments, the backend system is configured to transmit the downlink data to the SON at least partly based on at least one of input data obtained by the user input means of the wearable device and sensor data obtained by at least one sensor of the wearable device and which is received by the backend system from the SON.

In embodiments, the at least one of input data and sensor data obtained by the wearable device is transmitted to the backend system by the one hub node assigned by the SON to have the controller node role for the wearable device, wherein the node having the controller node role for a wearable device is responsible for determining and dynamically changing which of the nodes of the SON is currently having the only established short-range wireless connection between the wearable device and the SON.

In embodiments, the downlink data originating from the backend system is comprised in at least one of a configuration file which is continuously updated by the backend system and transmitted from the backend system to the SON, and control data transmitted downlink from the backend system to the SON.

In embodiments, the downlink data is received by the one hub node assigned by the SON to have the controller node role for the wearable device and responsible for determining and dynamically changing which only one of the nodes of the SON is currently having an established short-range wireless connection with the wearable device, and wherein the downlink data is shared with each of the other hub nodes by distribution.

In embodiments, each of the at least one wearable device is assigned one of the at least one hub node to have the controller node role for the respective wearable device and be configured for dynamically determining and changing which node is currently having a short-range wireless communication link established with the wearable device.

In embodiments, the technology disclosed provides a fast response yet robust solution for acting on an identified event by parallel redundant data transmission of event data representing or reflecting the event via both an established short-range wireless connection with one of the nodes and via short-range wireless broadcast messages from the personal device to the nodes which are within short-range wireless communication range.

Bluetooth is a short-range wireless technology standard that allows data to be exchanged between fixed and mobile devices. The technology uses short wavelength radio waves from 2.4 to 2.485 GHz. Unlike other forms of connectivity such as wi-fi or 4G, Bluetooth carries connections between devices and other devices as opposed to carrying data to and from the internet.

BLE stands for Bluetooth Low Energy and is a form of wireless communication designed especially for short-range communication. BLE is very similar to Wi-Fi in the sense that it allows devices to communicate with each other. However, BLE is meant for situations where battery life is preferred over high data transfer speeds. Wi-Fi uses multiple parts of the IEEE 802 protocol family and is designed to interwork seamlessly with its wired sibling Ethernet. Compatible devices can network through wireless access points to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 GHz (120 mm) UHF and 5 GHz (60 mm) SHF ISM radio bands; these bands are subdivided into multiple channels. Channels can be shared between networks but only one transmitter can locally transmit on a channel at any moment in time.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location, e.g. current room, for a personal device without any use of high energy-consuming GPS in the personal devices or iBeacon technology, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

The controller node, or a local or remote processing arrangement receiving the measured signals strengths from the controller node (and optionally also sensor data obtained by a sensor of the personal device), may then be configured to determine the current position or location for the personal device by comparing the signal strengths of the broadcast signals measured by the other nodes (and the controller node) and then determine the position or location based on the comparison. In embodiments, the position or location of the node measuring the highest signal strength for the broadcast signal is determined to be the position or location for the personal device, or the position or location for the personal device may be determined or calculated using signals strengths measured by a plurality of nodes, e.g. by using triangulation, or the position or location for the personal device may be determined or calculated using at least one signals strength of a broadcast signal and sensor data obtained by a sensor of the personal device, e.g. a motion sensor or a pressure sensor.

In certain embodiments, the current position or location for the personal device may at least partly be determined by a trained machine learning algorithm used by the controller node or a backend system communicatively coupled to the controller node, since machine learning is an efficient way of evaluating obtained sensor data and event data, e.g. in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically and continuously changing the connection node for a personal device, by measuring, by each of the network nodes which are within short-range communication range with the personal device, the signal strength of short-range wireless broadcast messages, e.g. Bluetooth broadcast messages, received from the personal device.

In certain embodiments, the setting of an alarm or the change of health state and/or alert state for the personal device may at least partly be determined by a trained machine learning algorithm used by the controller node or a backend system communicatively coupled to the controller node, since machine learning is an efficient way of evaluating sensor data in situations where large amounts of data is collected. The trained machine learning algorithm may then use at least the signal strength values received from the nodes (including the current controller node and the current connection node for the personal device if any of these nodes are within short-range wireless communication range with the personal device to receive the broadcasts) and/or movement sensor data detected by the personal device to determine that the connection node for the personal device should be changed.

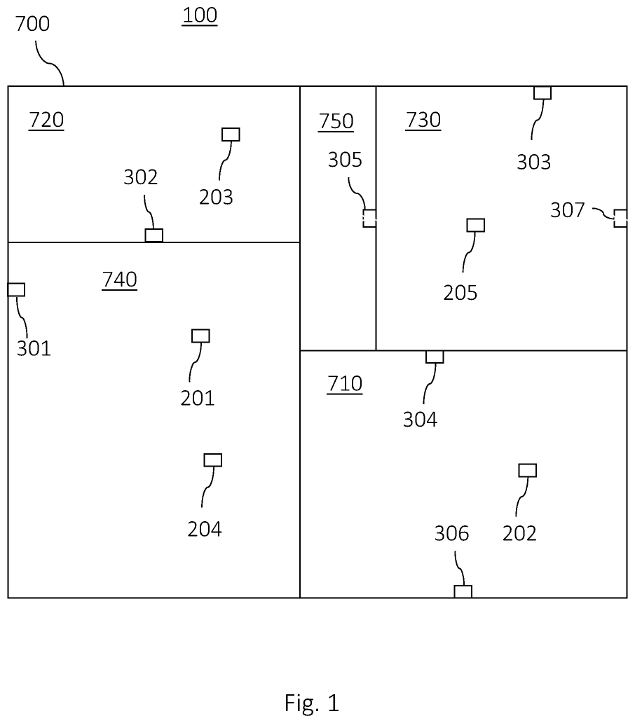

FIG. 1 schematically shows an example of a facility 700 where a system 100 comprising a number of network nodes (301, 302, 303, 304, 305, 306) has been installed, in accordance with one or more embodiments described herein. The illustrated system 100 comprises a plurality of wearable devices (201, 202, 203, 204, 205) carried by individuals and having short-range wireless communication capabilities. Such facility 700 may e.g. be an apartment where a monitored individual is residing. Such an apartment 700 may e.g. comprise a kitchen 710, a bathroom 720, a bedroom 730, a sitting room 740, and a hallway 750. The system 100 may in this case be used by relatives, home care companies or the like.

The facility 700 may also be other types of quarters where a monitored individual may be residing, such as e.g. a nursing home, where there may be a private space for the monitored individual, including e.g. a bedroom 730, as well as public spaces that are shared by a plurality of monitored individuals.

In this example embodiment of the technology disclosed, the network nodes with reference signs 301, 302 and 303 in FIG. 1 are hub nodes each configured to share data, including data associated with the wearable devices, with each other via established communication links between the nodes. The network nodes with reference signs 304, 305, 306 and 307 are satellite nodes configured to transmit short-range wireless broadcasts and to establish a short-range wireless communication link with a wearable device.

The wearable devices (201, 202, 203, 204, 205) are each connected to the network via an established short-range wireless communication link with one of the network nodes (301, 302, 303, 304, 305, 306, 307). The wearable devices (201, 202, 203, 204, 205) in FIG. 1 each have short-range wireless communication capabilities and comprises at least one of at least one sensor and a user input means. The wearable devices (201, 202, 203, 204, 205) are each configured to transmit short-range wireless broadcasts signals that may be received by any of the network nodes (301, 302, 303, 304, 305, 306, 307) that currently is within short-range wireless communication range with the wearable device transmitting the broadcasts.

The wearable device with reference sign 202 in FIG. 1 is currently connected to the network via a short-range wireless communication link with the satellite node with reference sign 306 and the wearable device with reference sign 201 in FIG. 1 is currently connected to the network via a short-range wireless communication link with the hub node with reference sign 301. The hub nodes (301, 302, 303) are each configured to both continuously share their data with other hub nodes and to establish short-range wireless communication links with each of the wearable devices.

The hub node with reference sign 301 in FIG. 1 is having the controller node role for the wearable device with reference sign 202, thereby being responsible for collecting sensor data and input data obtained by the wearable device 202, further being responsible for dynamically determining and changing which of the network nodes is currently having the only one established short-range wireless communication link with the wearable device 202.

In the example embodiment illustrated in FIG. 1, only the hub nodes (301, 302, 303) can have the roles of controller node for a wearable device (201, 202, 203, 204, 205). The hub node 301 in FIG. 1 can determine which of the hub nodes network nodes (301, 302, 303), including itself, is the connection node for the wearable device 202 by determining and changing which of the network nodes (301, 302, 303, 304, 305, 306), including itself, is currently having an established short-range wireless communication link with the wearable device 202. In this example, satellite node 306 is assigned by the controller node 301 to be connection node for the wearable device having the only one established short-range wireless communication link with the wearable device 202.

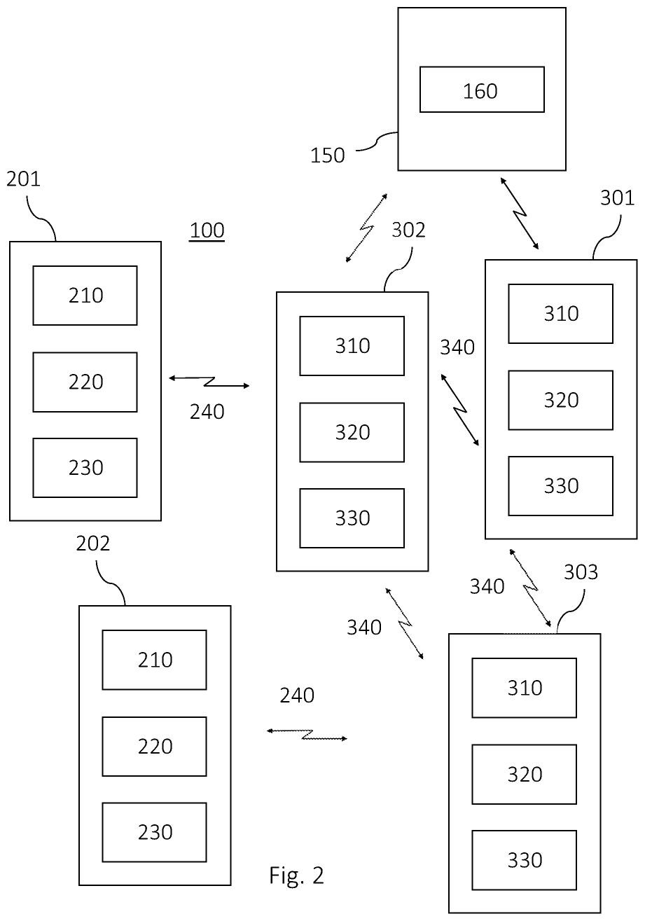

FIG. 2 is a schematic illustration of a system 100 for monitoring an individual within predetermined facilities 700, in accordance with one or more embodiments described herein. The illustrated system 100 comprises a plurality of wearable devices (201, 202), a plurality of network nodes (301, 302, 303), a processing arrangement 150, e.g. a remote processing arrangement such as a backend system, and an external system 170 for managing and an established call in which a wearable device 202 and a mobile communication device 190 associated with the wearable device 202 is participating. The wearable devices (201, 202) in FIG. 2 are wristbands worn by two separate individuals, e.g. a necklace or a clip worn by two patients in a nursing home 700.

The wearable devices (201, 202) illustrated in FIG. 2 each comprises user input means 220, and at least one sensor 230. The at least one sensor 230 may, for example, include a motion sensor, a temperature sensor, a health sensor such as a body temperature sensor, a pressure sensor and/or any other type of sensor and the user input means 220 may comprise a button or voice input means that the individual can press or activate to set off an alarm and/or transmit event data indicating the occurrence of a certain event. The illustrated network nodes (301, 302, 303) comprise processing means 310, a storage means 320, and a node communication interface 330. The illustrated processing arrangement 150 comprises a processing device 160, which may e.g. be comprised in a web server.

The hub node with reference sign 301 in FIG. 2 is having the controller node role for the wearable device with reference sign 202, thereby being responsible for collecting sensor data and input data obtained by the wearable device 202, further being responsible for dynamically determining and changing which of the network nodes is currently having the only one established short-range wireless communication link with the wearable device 202.

The controller node 302 for the wearable device 202 in FIG. 2 is further configured to receive downlink data related to the wearable device 202 from the backend system 150 and, in response to the downlink data is indicating changes in the behavior, movement pattern or medical condition of the individual carrying the wearable device 202, transmit control data to the wearable device 202 instructing the wearable device to active a sensor 203 of the wearable device 202.

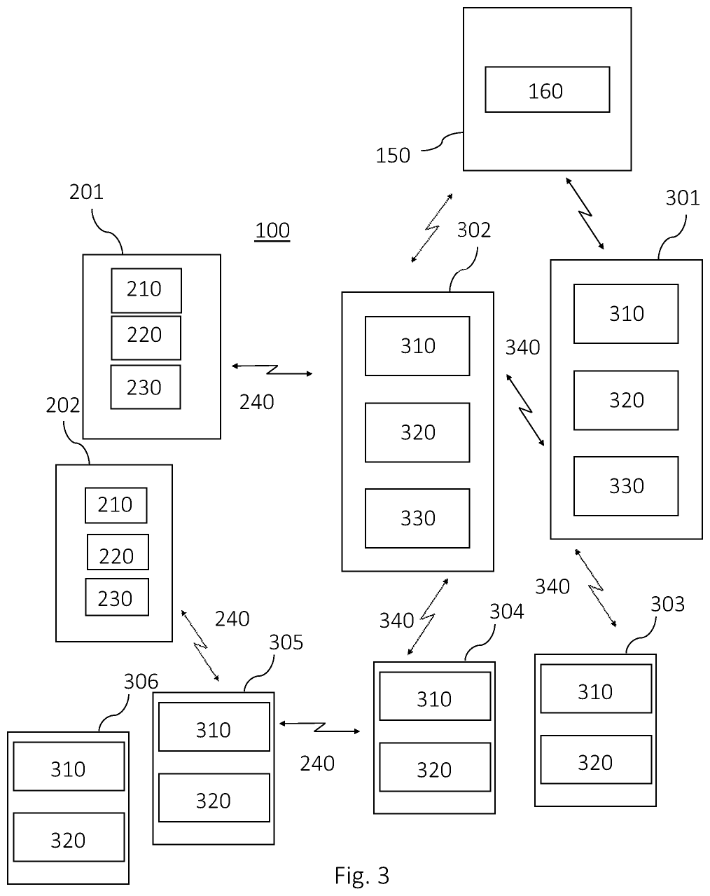

FIG. 3 is a schematic illustration of a system 100 for monitoring an individual within predetermined facilities 700, in accordance with one or more embodiments described herein. The example system 100 illustrated in FIG. 3 comprises two hub nodes comprising a speaker 310 and configured to have a controller node role for one of the wearable devices 201 and 202 and four satellite nodes comprising a speaker 310 and configured to both transmit short-range wireless broadcast signals and listen for short-range wireless broadcast signals transmitted by other devices and nodes.

The wearable devices (201, 202) illustrated in FIG. 3 each comprises a microphone 210, user input means 220, and at least one sensor 230. The at least one sensor 230 may, for example, include a motion sensor, a temperature sensor, a health sensor such as a body temperature sensor, a pressure sensor and/or any other type of sensor and the user input means 220 may comprise a button or voice input means that the individual can press or activate to set off an alarm and/or transmit event data indicating the occurrence of a certain event. The illustrated network nodes (301, 302, 303, 304, 305, 306) each comprises processing means 310 and a node communication interface 330. The hub nodes illustrated in FIG. 3 also comprise a storage means 320 for storing, for example, sensor data obtained by a wearable device the respective hub node is responsible for. The illustrated processing arrangement 150 comprises a processing device 160, which may e.g. be comprised in a web server.

In FIG. 3, the hub node 301 is the assigned controller node role for the wearable device 202. The wearable device 202 currently has an established short-range wireless communication link with satellite node 305. The hub node with reference sign 301 in FIG. 3 is having the controller node role for the wearable device with reference sign 202, thereby being responsible for collecting sensor data and input data obtained by the wearable device 202, further being responsible for dynamically determining and changing which of the network nodes is currently having the only one established short-range wireless communication link with the wearable device 202. The controller node 302 for the wearable device 202 in FIG. 2 is further configured to receive sensor data and/or input data obtained and transmitted by the wearable device 202 from or via the other network nodes (302,303,304,305,306) and, in response to the received sensor data and/or input data is indicating changes in the behavior, movement pattern or medical condition of the individual carrying the wearable device 202, transmit control data to the wearable device 202 instructing the wearable device to active a sensor 203 of the wearable device 202.

Information regarding the layout of the facilities 700, such as the plan of the different rooms and the location of the node modules 300, is preferably stored in the storage means 320. The storage means 320 does not have to be comprised in the network node—it can be comprised in another part of the system such as a locally-installed processing arrangement or a remote processing arrangement such as a backend system 150 or be a separate device or module.

According to embodiments of the technology disclosed, the network node with reference sign 301 in FIG. 3 is a hub node having the controller node role for the wearable device 202 and is therefore arranged to: receive status data, position data or signal strength values from the other hub nodes (302, 303) of the network, or sensor data, input data or event data originating from the at least one sensor 230 and/or user input means 220 of the wearable devices 202; determine whether the received data indicates a need for certain action associated with the wearable device 202, e.g. changing the health state for the individual wearing the personal device and/or alert state among a plurality of predetermined alert states. As an example, the alert states may comprise at least a FALL alert state, to be used if a probable fall has been detected for the monitored individual, and an OUT OF BED alert state, to be used if it has been detected that the monitored individual is probably getting out of bed. The hub nodes (301, 302, 303) in FIG. 3 receive sensor data and input data from the wearable device 202 which is received in broadcasts from the wearable device 202 by any satellite node (303, 304, 305, 306) within short-range communication range with the wearable device 202 and forwarded by the respective satellite node (303, 304, 305, 306) via short-range wireless transmissions to the hub nodes (301, 302, 303).

If weighing factors are used, these weighing factors may be determined by a machine learning system, since this is an efficient way of evaluating complex signals in situations where large amounts of data is collected.

One way of making such a determination is to determine a probability that an alert should be set based on the received event data and/or sensor data, and a probability that the determined location fulfils the predetermined location condition, and then combine these probabilities using weighing factors, which may e.g. be determined by a machine learning system.

The same applies to other combinations, such as e.g. the determination of the position and/or location for the individual wearing the personal device by involving also signals from other sensors of the personal device such as a movement sensor 230, and/or the setting of alerts based also on activity states. These determinations may also be done by determining probabilities and using weighing factors to combine them, which may e.g. be determined by a machine learning system.

In embodiments, the determining of a position and/or location for the individual wearing the personal device by the controller node (301) may e.g. be based on the received signal strength indication (RSSI) of short-range wireless broadcast signals transmitted by the wearable device 202 and which are received by the network nodes within short-range communication range with the personal device. The network nodes within short-range communication range then measure the RSSI of short-range wireless broadcast signals and forwards the RSSI to the controller node 301 for the wearable device 202. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by the wearable device 202 may be used by the controller node 301 assigned to the wearable device 202 for determining the current position and/or location of the wearable device 202 in connection with transmitting position or location data together with alert state data or when setting an alert or alarm for the individual wearing the wearable device 202.

The wearable devices (201, 202, 203, 204, 205) illustrated in the figures may also comprise other sensors 230, such as e.g. a pressure sensor, temperature sensor or medical sensor. It is known to use a pressure sensor in a personal device to detect a fall based on a change in pressure. However, a pressure sensor may also be involved in the determination of the location of the individual, e.g. when location data is transmitted together with health state data and/or alert state data to a local or remote processing arrangement 150, e.g. a backend system, configured to set an alert or an alarm. If the facilities 700 comprise several floors, and the network nodes are arranged in the ceilings or high up on the walls, the RSSI may give erroneous results due to the signals travelling between the floors. In this situation, a pressure sensor may be used to determine the floor on which the individual is located.

In order to improve the determination of the need for e.g. changing health state for the individual and/or setting an alert even further, the determination of the location may be improved by involving also signals from a movement sensor 230. The movement sensor 230 may e.g. be used as a pedometer, so that the signals from the movement sensor 230 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 230, it is likely that the determined location is incorrect. The at least one processing device comprised in at least one of a network node or in the backend system 160 may thus be arranged to determine the location of the wearable device 200 based also on an indication of the movement of the individual in relation to a previously determined location, based on sensor data from the at least one motion sensor 230.

The processing of the sensor data from the at least one sensor 230 may be done by a trained machine learning algorithm used by the controller node or a local or remote processing arrangement communicatively connected to the personal device. When many individuals are monitored using many personal devices 200, large amounts of sensor data is collected from the sensors 230 in these personal devices 200. Very efficient evaluation of this sensor data may be done using machine learning, in order to determine a suitable machine learning algorithm.

In the embodiments, the decision to active a second sensor may be based at least partly on obtained or received sensor data, e.g. obtained by a temperature sensor, pressure sensor or motion sensor of the same wearable device. In various embodiments, the actual decision to active a sensor of a wearable device may then be taken by the wearable device itself, a backend system or the network node having the controller node role for the wearable device.

The combination of motion information with location information improves the determination of changing connection node for the wearable device.

The determining of whether the received sensor data indicates a specific location among a plurality of predetermined location may e.g. be done by a trained machine learning algorithm of the controller node or a locally-installed processing arrangement or a remote processing arrangement such as a backend system communicatively connected to the controller node, since machine learning is an efficient way of evaluating complex signals in situations where large amounts of data is collected and moving patterns of the individuals wearing the personal devices are repetitive and/or predictable.

In embodiments, the distance between the personal device 200 and the at least one network node 300 may e.g. be determined based on the received or obtained signal strength indication (RSSI) of the short-range wireless signal transmitted by the personal device 200 and received by the plurality of network nodes, since the received signal strength will be lower if the distance increases. If the RRSI for more than one network node is determined, the location may be more exactly determined based on e.g. triangulation. Machine learning may also be used for this determination.

In embodiments, the at least one sensor 230 is an accelerometer. However, other types of motion sensors may also be used.

In embodiments, the personal device 200 is a wristworn device, such as e.g. a wristband. The personal device 200 may however be any type of device that may be worn by an individual, such as e.g. a necklace or a clip.

In embodiments, communication between the wearable device 200 and the at least one node 300 takes place using a personal device communication interface and a node communication interface. In embodiments, the technology disclosed may include continuously determining and updating an activity state for the monitored individual by continuously receiving sensor data from the personal device, e.g. health sensor data obtained by a medical sensor of the personal device.

The activity state may e.g. categorize the current activity of the individual. Activity states may e.g. be LYING IN BED, LYING ON COUCH, LYING ON FLOOR, SITTING, STANDING, WALKING, RUNNING, RIDING A WHEELCHAIR, WALKING UP/DOWN STAIRS, IN THE BATHROOM. There may also be a specific activity state for when the individual is not wearing the personal device, since no other activity state can then be set. There may be a specific sensor, such as e.g. a contact sensor, a temperature sensor or a heart rate sensor, for determining whether the individual is wearing the personal device. Such a sensor may of course also be used for other purposes.

In embodiments, the technology disclosed may include continuously determining and updating a health state for the monitored individual by continuously receiving sensor data from the personal device worn by the individual, e.g. health sensor data obtained by at least one medical sensor of the personal device.

The determining of the activity state and/or the health state of the individual wearing the personal device may e.g. be done by a trained machine learning algorithm, since machine learning is an efficient way of evaluating complex signals in situations where large amounts of data is collected.

In embodiments, the wearable device 200 may also comprise sensors, such as e.g. a motion sensor or pressure sensor. It is known to use a pressure sensor in a personal device to detect a fall based on a change in pressure.

However, a pressure sensor may also be involved in the determination, by the controller node, to activate a sensor of the personal device 200 carried by the individual. If the facilities 700 comprise several floors, and the network nodes 300 are arranged in the ceilings or high up on the walls, the RSSI may give erroneous results due to the signals travelling between the floors. In this situation, data obtained by a pressure sensor of the personal device received by the controller node may be used by the controller node to determine the floor on which the individual is located which, in turn, is an input factor to the step of determining, by the controller node for the personal device 200, whether to activate a sensor of the personal device 200.

In embodiments and in order to improve the determination of whether to activate a second sensor of a personal device 200, the determination of the location and/or whether to activate a sensor 230 may be improved by involving signals from a first sensor 230, e.g. in combination with input to a user input means of the personal device 200. The sensor 230 may e.g. be a movement sensor used as a pedometer, so that the signals from the movement sensor 230 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 230, it is likely that the determined location is incorrect. The at least one processing means 310 of a network node (301, 302, 303, 304, 305, 306) may thus be arranged to determine the location of the personal device 200 influencing the decision by the controller node, or processing arrangement, whether to change alert state and/or health state for the personal device 200 based also on an indication of the movement of the individual in relation to a previously determined location, based on sensor data from the at least one sensor 230.

The processing of the sensor data from the at least one sensor 230 may be done by a trained machine learning algorithm. When many individuals are monitored using many personal devices 200, large amounts of sensor data is collected from the sensors 230 in these personal devices 200. Very efficient evaluation of this sensor data may be done using machine learning, in order to determine a suitable machine learning algorithm.

The updating of the activity state and/or health state for the monitored individual may be incremental, so that only differences in relation to the previously determined activity state and/or health state need to be determined.

The determination of the location for the personal device 200 and/or the by the controller node to activate a sensor of the personal device 200 may e.g. be done by a trained machine learning algorithm. Machine learning is an efficient way of evaluating complex signals in situations where large amounts of data is collected.

When the determining of whether to change alert state and/or health state is based on received sensor data indicates a certain location and whether the determined location fulfils a predetermined location condition, the predetermined location condition may be an absolute condition, such that if the determined location does not fulfil the predetermined location condition, the controller node will, or will not, determine to change alert state and/or health state for the personal device 200. However, there may instead be weighing factors, so that if the received sensor data strongly indicates a specific location, and/or the determined location does not strongly indicate a specific location (that does not fulfil the predetermined location condition), the controller node will, or will not, determine to change alert state and/or health state for the personal device 200.

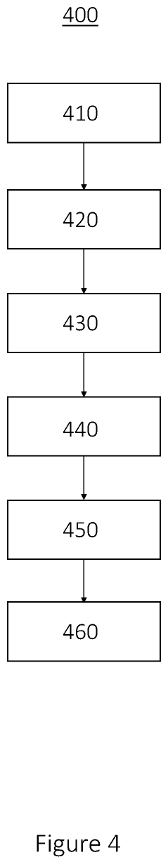

FIGS. 4 to 6 show flow diagrams of various example methods according to the technology disclosed for monitoring an individual within predetermined facilities in order to determine whether there is a need for activating a sensor of a wearable device, e.g. in response to a changed health state or changed position and/or body position for the individual and/or setting an alert, in accordance with one or more embodiments described herein.

FIG. 4 schematically illustrates embodiments of the technology disclosed including a method (400) in a system including a network comprising at least three network nodes and at least one wearable device having short-range wireless communication capabilities and comprising at least one sensor and a user input means, the method comprising:
a. assigning (410) one of the network nodes to be responsible for a particular wearable device in collecting sensor data and/or input data obtained and transmitted by the wearable device to the network nodes of the network;
b. continuously receiving (420), by the network node responsible for a wearable device, sensor data and/or user input data obtained by the at least one sensor and a user input means of the wearable device;
c. determining (430), by the network node responsible for the wearable device, that received sensor data and/or user input data is indicating to the network node certain changes in the behavior or medical condition of the individual carrying the wearable device and instructions that a sensor of the wearable device is to be activated;
d. transmitting (440), by the network node to the wearable device, control data instructing the activation of a sensor of the wearable device, and
e. receiving (450), by the wearable device, said control data transmitted by the network node; and
f. activating (460), by the wearable device and in response to the received control data, the currently inactive sensor of the wearable device.

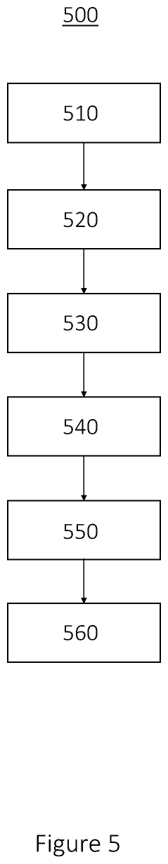

FIG. 5 schematically illustrates embodiments of the technology disclosed including a method (500) a system including a backend system, a network comprising at least three network nodes and at least one wearable device having short-range wireless communication capabilities and comprising at least one sensor and a user input means, the method comprising:
a. assigning (510) one of the network nodes to be responsible for a wearable device in collecting sensor data and/or input data obtained and transmitted by the wearable device to the network nodes of the network;
b. continuously receiving (520), by the network node responsible for a wearable device, sensor data and/or user input data obtained by the at least one sensor and a user input means of the wearable device;
c. transmitting (530), by the node responsible for the wearable device to the backend system, data based on the received sensor data and/or user input data;
d. transmitting (540), by the backend system, downlink data to the SON, wherein the downlink data is based on sensor data and/or user input data received from the node responsible for the wearable device;
e. receiving (550), by the node responsible for the wearable device, the downlink data from the backend system, and in response to the downlink data from the backend system is containing at least one of data indicating to the responsible network node certain changes in the behavior or medical condition of the individual carrying the wearable device and instructions that a sensor of the wearable device is to be activated, the activation of a sensor of the wearable device by:
   f. transmitting (560), by the node responsible for the wearable device to the wearable device, control data instructing the wearable device to active a sensor.

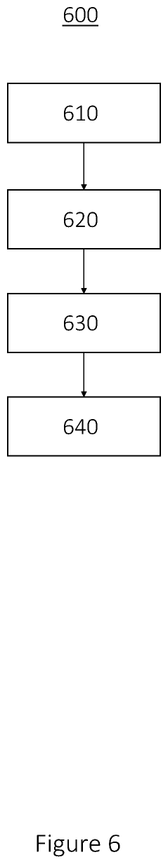

FIG. 6 schematically illustrates embodiments of the technology disclosed including a method (500) a system including a backend system, a network comprising at least three network nodes and at least one wearable device having short-range wireless communication capabilities and comprising at least one sensor and a user input means, the method comprising:
a. continuously obtaining (610), by the at least one of at least one sensor and a user input means of a wearable device, sensor data and/or user input data;
b. transmitting (620), by a network node, control data to the wearable device instructing the wearable device to activate at least one of its sensors, wherein said transmission is in response to obtaining certain values of received sensor data and/or user input data:
   c. activating (630), by the control system of the wearable device and in response to receiving said control data, a sensor of the wearable device to begin obtaining sensor data, and
d. continuously transmitting (640) sensor data obtained by the activated sensor over an established short-range wireless communication link so that the sensor data can be obtained by the network.

1. A system for monitoring individuals including a backend system, a self-organizing network (SON) comprising at least three network nodes communicatively connected to each other via established communication links and at least one wearable device comprising at least one at least one sensor, and configured to transmit short-range wireless broadcast signals via its short-range wireless transmitter and temporarily listen for responses to the short-range wireless broadcast signals transmitted by themselves,
wherein each wearable device is assigned one network node to have the controller node role for the wearable device in that the controller node for a wearable device is configured to control and dynamically change which only one of the network nodes that currently has a short-range wireless connection with the wearable device,
wherein the network node having the controller node role for a wristband is further configured to receive and collect input data and/or sensor data for the wearable device received from the other network nodes, and
wherein each wearable device is configured to receive and/or obtain at least one of control data, input data and sensor data and,
in response to receiving at least one of control data instructing the wearable device to active an inactive sensor or group of sensors of the wearable device, and input data and/or sensor data indicating to the wearable device at least one of an alert state, a changed movement pattern or position, a changed physical condition, a changed health state and a changed physical orientation of the individual wearing the wearable device, activate an inactive sensor or group of sensors of the wearable device to start obtaining sensor data from the activated sensor or group of sensors and then transmit the obtained sensor data over the established one short-range wireless connection with the only one network node so that the sensor data is received by the network node having the controller node role for the wearable device.

2. The system according to claim 1, wherein the wearable device is configured to activate a second sensor or second group of sensors of the wearable device in response to at least one of input data and sensor data obtained by a first sensor or first group of sensors of the wearable device different from the second sensor or second group of sensors is indicating an alert state, a changed movement pattern or position, a changed physical condition, a changed health state and a changed physical orientation of the person wearing the wearable device.

3. The system according to claim 1, wherein the wearable device is configured to activate a second sensor or second group of sensors of the wearable device in response to received control data originating from the controller node for the wearable device and which transmission from the controller node was triggered by at least one of input data and sensor data obtained by a first sensor or first group of sensors of the wearable device is indicating at least one of an alert state, a changed movement pattern or position, a changed physical condition, a changed health state and a changed physical orientation of the person wearing the wearable device.

4. The system according to claim 1,
wherein the hub node having the controller node role for a wearable device and being responsible for the wearable device is further configured to receive and collect input data and/or sensor data obtained by the input data and/or sensor data of the wearable device and, in response receiving input data and/or sensor data indicating at least one of an alert state, a changed movement pattern or position, a changed physical condition, a changed health state and a changed physical orientation of the person wearing the wearable device:
transmit control data to the wearable device instructing the wearable device to activate the sensor of the wearable device.

5. The system of claim 1,
wherein the hub node having the controller node role for the wearable device is further configured to receive and collect at least one of input data and sensor data obtained by an input means and/or sensor of the wearable device and, in response to the receiving input data and/or sensor data indicating at least one of an alert state, a changed movement pattern or position, a changed physical condition, a changed health state and a changed physical orientation of the person wearing the wearable device:
transmit control data to the wearable device instructing the wearable device to at least one of:
start discarding sensor data obtained by a sensor of the wearable device,
change the amount of discarded sensor data obtained by a sensor of the wearable device, and
increase or decrease the sampling rate or interval for at least one sensor of the wearable device.

6. The system according to claim 1,
wherein the activated sensor or group of sensors is in sleep mode operation before being activated by the wearable device.

7. The system according to claim 1,
wherein the hub node having the controller node role for a wearable device is configured to, in response to receiving downlink data from the backend system which indicates at least one of an alert state, a changed physical condition, a changed health state, a changed physical orientation of the person wearing the wearable device and that a call should be established with the wearable device: transmit control data to the wearable device instructing the wearable device to activate the inactive sensor or group of sensors of the wearable device.

8. The system according to claim 7, wherein the downlink data is based on at least one of input data and sensor data obtained by the wearable device which is transmitted to the backend system by the one hub node assigned by the SON to have the controller node role for the wearable device.

9. The system according to claim 7,
wherein the downlink data from the backend system is comprised in at least one of a configuration file which is continuously updated by the backend system and which is based on input data and/or sensor data received from the SON and then transmitted from the backend system to the SON, and control data transmitted downlink from the backend system to the SON which is based on input data and/or sensor data received from the SON and which is adapted for instructing the hub node having the controller node role for the wearable device to trigger the transmission of the control data to the wearable device instructing the wearable device to activate the sensor of the wearable device.

10. The system according to claim 1,
wherein the hub node having the controller node role for a wearable device is further configured to, in response to receiving downlink data originating from the backend system and which indicates at least one of an alert state, a changed physical condition, a changed health state, a changed physical orientation of the person wearing the wearable device and that a call should be established with the wearable device, transmit control data instructing the wearable device to activate the inactive sensor or group of sensors of the wearable device.

11. The system according to claim 1,
wherein the backend system is configured to transmit downlink data to the SON at least partly based on at least one of input data obtained by the input device of the wearable device and sensor data obtained by at least one sensor of the wearable device and which is received by the backend system from the SON.

12. The system according to claim 1,
wherein the system comprises at least two hub nodes and each of the at least one wearable device is assigned, by the SON, one of the hub nodes to have the controller node role for the respective wearable device and be configured for dynamically determining and changing which node is currently having the only one short-range wireless communication link established between the SON and the wearable device it is responsible for.

13. The system according to claim 1,
wherein the SON comprises at least two hub nodes each communicatively coupled to the other hub nodes through established connections and configured to receive at least one of input data and sensor data from the at least one wearable device and then share the received data with other hub nodes by distributing the data over the established connections links.

14. The system according to claim 1,
wherein the SON comprises at least three satellite nodes each configured to establish a short-range wireless connection with a wearable device in response to instructions received from the node having the controller node role for the wearable device, and to forward input data and/or sensor data received from the wearable device in short-range wireless broadcast transmissions received by other network nodes within short-range communication range.

15. A method in a system for monitoring individuals and including a network comprising at least three network nodes, a backend system and at least one wearable device having short-range wireless capabilities and comprising at least one sensor, the method comprising:
a. assigning one of the network nodes to be responsible for a particular wearable device in collecting sensor data and/or input data obtained and transmitted by the wearable device to the network nodes of the network;
b. continuously receiving, by the network node responsible for a wearable device, sensor data and/or user input data obtained by the at least one sensor and a user input means of the wearable device;
c. receiving, by the wearable device, at least one of control data instructing the wearable device to active an inactive sensor or group of sensors of the wearable device, and input data and/or sensor data indicating to the wearable device at least one of an alert state, a changed movement pattern or position, a changed physical condition, a changed health state and a changed physical orientation of the individual wearing the wearable device; and
d. activating, by the wearable device, the currently inactive sensor of the wearable device, wherein said activating of the currently inactive sensor is triggered by and/or in response to at least one of receiving control data instructing the wearable device to active an inactive sensor or group of sensors of the wearable device and receiving input data and/or sensor data indicating to the wearable device at least one of an alert state, a changed movement pattern or position, a changed physical condition, a changed health state and a changed physical orientation of the individual wearing the wearable device.

16. The method according to claim 15, said method is further comprising:
a. transmitting, by the network node to the wearable device, control data instructing the activation of a sensor of the wearable device;
b. receiving, by the wearable device, said control data transmitted by the network node; and
c. activating, by the wearable device and in response to the received control data, the currently inactive sensor of the wearable device.

17. The method according to claim 15, wherein said determining is performed by the network node responsible for the wearable device, said method further comprising the steps of:
a. transmitting, by the node responsible for the wearable device to the backend system, data based on the received sensor data and/or user input data;
b. transmitting, by the backend system, downlink data to the SON, wherein the downlink data is based on the sensor data and/or user input data received from the node responsible for the wearable device;
c. receiving, by the node responsible for the wearable device, the downlink data from the backend system,
and in response to the downlink data from the backend system is containing at least one of data indicating to the responsible network node certain changes in the behavior or medical condition of the individual carrying the wearable device and instructions that a sensor of the wearable device is to be activated, the activation of a sensor of the wearable device by:
d. transmitting, by the node responsible for the wearable device to the wearable device, control data instructing the wearable device to active a sensor.
